(19) United States
(12) Patent Application Publication
(10) Pub. No.: US 2023/0050202 A1
(43) Pub. Date: Feb. 16, 2023

(54) ENGINEERED FIBROBLASTS AS CELL THERAPY TO TREAT CANCER VIA TUMOR STROMA STABILIZATION

(71) Applicant: The Johns Hopkins University, Baltimore, MD (US)

(72) Inventors: Yun Chen, Pikesville, MD (US); Wei-Hung Jung, Baltimore, MD (US); Michael J. Betenbaugh, Baltimore, MD (US); Franck Housseau, Ashburn, VA (US)

(21) Appl. No.: 17/792,477

(22) PCT Filed: Jan. 15, 2021
(86) PCT No.: PCT/US2021/013650

§ 371 Date: Jul. 13, 2022

Related U.S. Application Data
(60) Provisional application No. 62/961,838, filed on Jan. 16, 2020.

(51) Int. Cl. C12N 5/077; C12N 9/10; C12N 15/90; C12N 15/11; C12N 9/22; A61K 35/33; A61P 35/00

(52) U.S. Cl. CPC C12N 5/0656; C12N 9/1044; C12Y 203/02013; C12N 15/907; C12N 15/11; C12N 9/22; A61K 35/33; A61P 35/00; C12N 2310/20; C12N 2800/80; C12N 2502/30; C12N 2510/00; C12N 2513/00

(57) ABSTRACT

The disclosure is directed to compositions and methods comprising genetically engineered fibroblasts for inhibiting progression of a cancerous tumor.

### STATEMENT OF RELATED APPLICATIONS

This application is a national phase application under 35 U.S.C. § 371 of PCT International Application No. PCT/US2021/013650, filed Jan. 15, 2021, which claims priority to U.S. Provisional Patent Application No. 62/961,838, filed on Jan. 16, 2020, which is hereby incorporated by reference for all purposes as if fully set forth herein.

### BACKGROUND

Cancer cells, extracellular matrix (ECM), and carcinoma-associated fibroblasts (CAFs) are three critical factors contributing to tumor progression in many cancer types, including breast cancer. CAFs are often associated with the development of high-grade malignancies of poor prognoses, because CAFs secrete metastasis-promoting cytokines and abnormally deposit collagen, facilitating integrin-dependent cancer invasion and forming hindrance of anti-cancer drug delivery. It has become clear that the surrounding environment of cancer, such as ECM and CAFs, plays an important role in the occurrence and development of cancer. Thus, targeting the surrounding environment of cancer has attracted attention as a potential therapeutic strategy for cancer.

### BRIEF SUMMARY OF THE INVENTION

The disclosure provides a composition comprising fibroblasts that have been genetically engineered to (a) express one or more genes that are downregulated in carcinoma-associated fibroblasts (CAFs) and/or (b) silence one or more genes that are upregulated in cancer CAFs.

The disclosure also provides method of inhibiting progression of a cancerous tumor, which method comprises: (a) genetically engineering a population of fibroblasts obtained from a subject to (a) express one or more genes that are downregulated in carcinoma-associated fibroblasts (CAFs) and/or (b) silence one or more genes that are upregulated in cancer CAFs; and (b) administering the genetically engineered population of fibroblasts to the microenvironment of a cancerous tumor present in the subject, whereupon the one more genes downregulated in CAFs are expressed and/or the one or more genes upregulated in cancer CAFs are silenced in the microenvironment of the cancerous tumor, thereby inhibiting progression of the cancerous tumor in the subject.

The disclosure also provides method of inhibiting extracellular matrix (ECM) remodeling in the microenvironment of a cancerous tumor, which comprises administering to the microenvironment of a cancerous tumor a composition comprising one or more extracellular matrix crosslinking proteins and a pharmaceutically acceptable carrier, whereby ECM remodeling in the microenvironment of the cancerous tumor is inhibited.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWING(S)

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic diagram illustrating certain aspects of the invention.

FIGS. 2A-2G illustrate that coculturing early-stage breast cancer cells with fibroblasts causes ECM fibrils alignment and induces CAF-associated phenotypes. FIG. 2A is a schematic depicting the model system in which tumor spheroids are embedded in ECM with fibroblasts in the periphery. FIG. 2B shows that ECM fibrils were radially aligned in the coculture of 67NR spheroid and GFP-transfected fibroblasts (right), while the fibrils remained isotropic in the coculture containing EpH4-Ev and GFP-transfected fibroblasts (left). FIG. 2C shows that fibroblasts (green) cocultured with an EpH4-Ev or 67NR spheroid migrated through the ECM over time. The dotted yellow lines trace the boundary of spheroids. The yellow arrows indicate the same fibroblast at different time points. The red arrows indicate local fibril alignment. FIG. 2D shows that fibroblasts cocultured with 67NR spheroids (bottom) showed persistent migration toward the spheroid (right half of the rose chart), but not the ones cocultured with EpH4-Ev (top). EpH4-Ev: N=61, 67NR: N=61. FIG. 2E shows that fibroblasts cocultured with 67NR spheroids showed higher velocity. EpH4-Ev: N=72, 67NR: N=61. FIGS. 2F and 2G show that CAF markers FSP1 and αSMA exhibited higher expression in fibroblasts cocultured with 67NR spheroids. The fibroblasts within and outside the 300-μm perimeter from the spheroid were analyzed separately. All data were normalized to the average from the EpH4-Ev>300 μm. EpH4-Ev: N=81 (<300 μm) and 83 (>300 μm), 67NR: N=83 (<300 μm) and 76 (>300 μm). Scale bars: 200 μm (FIG. 2B), 100 μm (FIGS. 2C and 2F).

FIGS. 3A-3F illustrate that early-stage cancer cells align the ECM fibrils and facilitate faster diffusion towards stroma. FIGS. 3A and 3B include time-lapse images showing that ECM fibrils were reoriented to be aligned over time in the 67NR spheroid-only, but not EpH4-Ev spheroid-only culture. Yellow arrowheads indicate the aligned fibrils. FIG. 3C shows results of fibril coherence analysis and that in 67NR spheroid-only cultures, higher fibril alignment increased over time within and outside the 300-μm perimeter from the spheroid. FIG. 3D shows quantification of the difference between fibril coherence at 3-hour and 20-hour incubation. ECM fibril alignment was increased by 67NR spheroids. For FIGS. 3C and 3D, EpH4-Ev: N=12 (<300 μm) and 10 (>300 μm), 67NR: N=13 (<300 μm) and 10 (>300 μm). FIG. 3E is a schematic of the line FRAP experiment depicting that 2,000 kDa dextran-FITC is added to the spheroid-embedded ECM to be photobleached in the shape of two orthogonally oriented stripes: one along to the fibrils originated from the spheroid, the other perpendicular to the first stripe. The red arrows indicate the radial diffusion relative to the spheroid. If the diffusion in the radial direction is significantly faster, the recovery in the orthogonal stripe would be faster, because there is a shorter gap for the radially diffusing dextran-FITC to fill. FIG. 3F shows that diffusion enhancement time τ_{DE} was increased by 67NR spheroids. EpH4-Ev: N=25, 67NR: N=34. Scale bar: (a) 200 μm.

FIGS. 4A-4G illustrate that fibril alignment facilitates anisotropic diffusion of exosomes and increases CAF marker expression. FIG. 4A is a schematic showing that the magnet pair (inset) was used to align ECM fibrils mixed with paramagnetic nanoparticles. Supernatant from 67NR-cultured medium and DMEM were added to the reservoirs. FIG. 4B shows that fibroblasts were oriented correspondingly after being cultured in the magnetically aligned ECM for two days. FIG. 4C shows that magnetically aligned ECM fibrils gave higher coherence scores. Control: N=100. Aligned ECM: N=93. FIG. 4D shows that diffusion enhancement was observed in magnetically aligned fibrils in the aligned direction. Control: N=10. Aligned ECM: N=15. FIGS. 4E and 4F show that FSP1 and αSMA expression was higher in fibroblasts grown in magnetically aligned ECM. FIG. 4G shows quantification of FSP1 and αSMA expression at various distances away from the 67NR-supernatant supplemented reservoir. All data were normalized to the average of the <1000 μm group. For FSP1, control: N=25 (<1000 μm), 15 (1000-2000 μm), 21 (2000-3000 μm), and 21 (3000-4000 μm), aligned ECM: N=27 (<1000 μm), 13 (1000-2000 μm), 9 (2000-3000 μm), and 15 (3000-4000 μm). For αSMA, control: N=25 (<1000 μm), 15 (1000-2000 μm), 21 (2000-3000 μm), and 20 (3000-4000 μm), aligned ECM: N=26 (<1000 μm), 13 (1000-2000 μm), 9 (2000-3000 μm), and 12 (3000-4000 μm). Scale bars: 200 μm (FIG. 4B), 25 μm (FIGS. 4E and 4F).

FIGS. 5A-5F illustrate that ECM crosslinking reduces fibril alignment and CAF induction. FIGS. 5A and 5B show that 67NR-embedded ECM treated with 0.5 mM genipin showed reduced alignment. Control: N=20, 0.5 mM Genipin: N=35. FIG. 5C shows that diffusion enhancement was not observed in crosslinked ECM. Control: N=25, 0.5 mM Genipin: N=16. FIG. 5D is a graph of rheological measurement which showed that 0.5 mM genipin-treated ECM exhibits comparable shear moduli as the uncrosslinked control. Control: N=3, 0.5 mM Genipin: N=3, 1 mM Genipin: N=2. FIGS. 5E and 5F show that FSP1 and αSMA expression in fibroblasts from crosslinked cocultures was reduced. All data were normalized to the average from the control>300 μm. For FSP1, control: N=54 (<300 μm) and 39 (>300 μm), 0.5 mM Genipin: N=20 (<300 μm) and 16 (>300 μm). For αSMA, control: N=15 (<300 μm) and 15 (>300 μm), 0.5 mM Genipin: N=45 (<300 μm) and 45 (>300 μm). Scale bars: 50 μm (FIG. 5A), 100 μm (FIG. 5E).

FIGS. 6A-6I illustrate that fibril alignment and subsequent CAF induction by cancer cells is force-dependent. FIGS. 6A and 6B show results of traction force microscopy indicating that expressing dominant negative RhoA, RhoA^{T19N}, reduced traction forces generated by 67NR spheroids. The shape of the spheroid is delineated by the red contour. EpH4-Ev: N=7, 67NR: N=6, 67NR-RhoA^{T19N}: N=9. FIGS. 6C and 6D show that 67NR-RhoA^{T19N}-embedded ECM exhibited reduced fibril alignment. 67NR: N=13 (<300 μm) and 10 (>300 μm), 67NR-RhoA^{T19N}: N=11 (<300 μm) and 11 (>300 μm). FIG. 6E shows that 67NR-RhoA^{T19N}-embedded ECM exhibited no diffusion enhancement. 67NR: N=34, 67NR-RhoA^{T19N}: N=43. FIGS. 6F and 6G show that fibroblasts cocultured with 67NR-RhoA^{T19N} spheroids exhibited reduced FSP1 and αSMA expression. All data were normalized to the average from the 67NR>300 μm. For FSP1, 67NR: N=83 (<300 μm) and 76 (>300 μm), 67NR-RhoA^{T19N}: N=10 (<300 μm) and 15 (>300 μm). For αSMA, 67NR: N=83 (<300 μm) and 76 (>300 μm), 67NR-RhoA^{T19N}: N=9 (<300 μm) and 15 (>300 μm). FIGS. 6H and 6I illustrate the proposed model. Scale bars: 100 μm (FIGS. 6A, 6C, and 6F).

FIG. 7 is a schematic diagram illustrating the workflow of engineering STAR fibroblasts. The dermal fibroblasts will be first isolated from the mouse with CRC. The isolated fibroblasts will be then genetically engineered to suppress the ECM remodeling. Finally, the STAR fibroblasts will be injected into mice with CRC as cell therapy.

FIG. 8 is a schematic diagram illustrating known differences in normal colonic fibroblast and CAFs.

FIGS. 9A and 9B illustrate that prototype STAR fibroblasts with RhoA signaling inhibition described in Example 9 prevent TGFβ1-mediated CAF induction. FIG. 9A includes images showing that the expression of CAF marker αSMA (green) did not increase in the prototype STAR fibroblasts after TGFβ1 treatment. Scale bar: 30 μm. FIG. 9B is a graph which quantifies the data of FIG. 9A.

FIGS. 10A and 10B illustrate that co-culture of prototype STAR fibroblasts exhibiting RhoA signaling inhibition with human lung cancer spheroid suppresses cancer cell invasion. FIG. 10A includes representative images showing the spreading areas of cancer cell invasion indicated by the traced line, which represents the degree of cancer invasion. Scale bar: 200 μm. FIG. 10B is a graph which quantifies the data of FIG. 10A.

FIGS. 11A and 11B illustrate that the prototype STAR fibroblasts described in Example 9 suppress cancer cell proliferation. FIG. 11 includes a series of images showing expression of the cell proliferation marker Ki67 in cancer cells co-cultured with prototype STAR fibroblasts. Scale bar: 100 μm. FIG. 11B is a graph illustrating the percentage of Ki67-positive A549 cancer cells co-cultured with the prototype STAR fibroblasts.

FIGS. 12A and 12B illustrate that prototype STAR fibroblasts with RhoA signaling inhibition potentiate the killing efficacy of cancer cells by anti-cancer treatment. FIG. 12A is a series of representative images showing viability of A549 cancer cells examined after another 24 hours of co-culture with STAR fibroblasts by staining with EthD-1 dye (red) and Hoechst (green). Red fluorescence indicates dead cells, and green indicates live cells. Scale bar: 100 μm. FIG. 12B is a graph which quantifies the data of FIG. 12A.

FIG. 13A is a graph showing that TGFβ1-treated prototype STAR fibroblasts described in Example 9 did not result in increased cancer invasion. FIG. 13B is a graph showing that TGFβ1-treated prototype STAR fibroblasts described in Example 9 did not result in increased cancer proliferation.

### DETAILED DESCRIPTION OF THE INVENTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

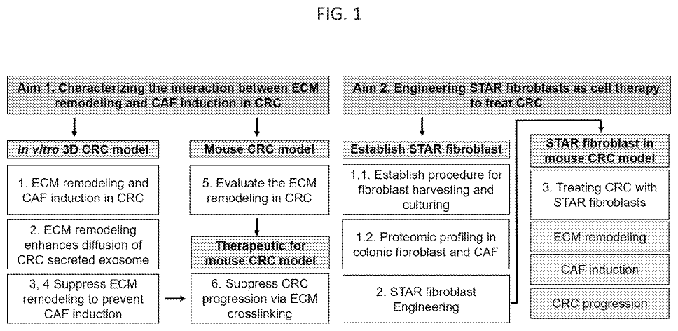

The disclosure provides an extracellular matrix (ECM)-crosslinking treatment to suppress carcinoma-associated fibroblast (CAF)-mediated tumor progression, as illustrated in FIG. 1. In particular, described herein is a fibroblast-based cell therapy, referred to as “stabilization against remodeling (STAR) fibroblasts,” to treat pre-metastatic cancer. In some embodiments, normal fibroblasts may be harvested from the same patient or animal and genetically engineered to secrete ECM crosslinking enzymes and to restore the normal ECM architecture. The efficacy of the STAR fibroblasts may be tested in a mouse model of colorectal cancer, breast cancer, or melanoma, by examining the ECM remodeling, CAF induction, and disease progression metrics, as shown in FIG. 1.

In some embodiments, the disclosure provides a composition comprising fibroblasts that have been genetically engineered to (a) express one or more genes that are downregulated in carcinoma-associated fibroblasts (CAFs) and/or (b) silence one or more genes that are upregulated in cancer CAFs. The terms “genetically engineer,” “genetically modify,” and “genetically manipulate,” may be used interchangeably herein to refer to the direct artificial manipulation, modification, or recombination of DNA or other nucleic acid molecules in order to modify an organism or population of organisms. More specifically, genetic engineering encompasses the set of technologies used to change the genetic makeup of cells, including the transfer of genetic material within and across species boundaries to produce improved or novel organisms. Likewise, the terms “non-naturally occurring” or “engineered” are used interchangeably herein and indicate the involvement of the hand of man. The terms, when referring to nucleic acid molecules or polypeptides, mean that the nucleic acid molecule or the polypeptide is at least substantially free from at least one other component with which they are naturally associated in nature and as found in nature.

Fibroblasts are the most common cells of connective tissue in animals. Fibroblasts produce the ECM's structural proteins (e.g., fibrous collagen and elastin), adhesive proteins (e.g., laminin and fibronectin), and ground substance (e.g., glycosaminoglycans, such as hyaluronan and glycoproteins). However, fibroblasts also play various additional roles beyond ECM production. For example, fibroblasts serve pivotal roles in ECM maintenance and reabsorption, wound healing, inflammation, angiogenesis, cancer progression, and in physiological as well as pathological tissue fibrosis. Fibroblasts are mesenchymal cells derived from the embryonic mesoderm tissue, and they are not terminally differentiated. They can be activated by a variety of chemical signals that promote proliferation and cellular differentiation to form myofibroblasts with an up-regulated rate of matrix production (see, e.g., R. T. Kendall and C. A. Feghali-Bostwick, Front. Pharmacol., 27 May 2014; doi.org/10.3389/fphar.2014.00123).

The term “carcinoma-associated fibroblasts,” as used herein, refers to spindle-shaped cells that build up and remodel the extracellular matrix (ECM) structure. CAFs are one of the most dominant components in the tumor stroma (Kalluri R., Nat Rev Cancer. 2016; 16:582-98). Detection of carcinoma-associated fibroblasts (CAFs) in cancer patients is associated with poor prognosis. CAFs remodel the tumor microenvironment to promote efficient metastasis. The term “tumor microenvironment (TME),” as used herein, refers to a multicellular system composed of tumor cells themselves, as well as cells from mesenchymal, endothelial, and hematopoietic origins arranged in the extracellular matrix (ECM), which interact closely with tumor cells and contribute to tumorigenesis. The tumor-TME crosstalk regulates, either positively or negatively, cancer progression (Quail D F, Joyce J A., Nat Med. 2013; 19:1423-37). CAFs also secrete cytokines to promote proliferation and to resist apoptosis in cancer cells. Although direct incubation of secreted CAF-promoting factors from breast cancer cells with normal fibroblasts (NFs), such as cytokines (e.g. TGF-β), microRNAs, and exosomes containing cytokine and/or microRNA, were shown to induce CAF phenotypes in vitro, it is not known how CAF-promoting factors reach stromal NFs in vivo. CAF induction can occur at early cancer stages, when cancer cells are not yet metastatic, secrete low metalloproteases (MMPs), and are still restricted by the intact ECM surrounding the tumor. It is not yet known how cancer cells overcome the diffusion barrier imposed by the dense ECM fibrils in the tumor microenvironment. As described in the Examples, a 3D coculture system was developed to mimic the microenvironment of early-stage breast cancer, which shows that cancer cells align the ECM fibrils and induce CAF phenotypes in NFs. It was also found that the fibril alignment is a force-dependent process and results in enhancing the diffusion of CAF-promoting exosomes secreted by cancer cells. By disrupting RhoA signaling or crosslinking ECM fibrils, ECM fibril alignment and diffusion enhancement is suppressed, and CAF induction reversed. It has been hypothesized, therefore, that force generation plays an important role during early-stage cancer development by remodeling tumor microenvironment.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholine nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or a RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

It will be appreciated that expression of a gene is downregulated if the expression is reduced by at least about 20% (e.g., 25%, 30%, 35%, 40%, 45%, 50%, 55%, 60%, 65%, 70%, 75%, 80%, 85%, 90%, 95%, 99% or more) as compared to a reference level or control. Expression of a gene is upregulated if the expression is increased by at least about 20% (e.g., 25%, 30%, 35%, 40%, 45%, 50%, 55%, 60%, 65%, 70%, 75%, 80%, 85%, 90%, 95%, 99% or more) as compared to a reference level or control.

Expressing one or more genes that are downregulated in cancer associated fibroblasts (CAFs) may be accomplished using any methods known in the art for artificially inducing gene expression. Such methods include, but are not limited to, gene transfer into cells via “transfection,” “transformation,” or “transduction.” The terms “transfection,” “transformation,” or “transduction,” as used herein, refer to the introduction of one or more exogenous polynucleotides into a host cell using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors also may be used to introduce exogenous nucleic acid sequences into host cells, many of which are commercially available.

The terms “silence” and “silence expression,” as used herein, refer to inhibition of expression of a particular gene. The degree of inhibition may be partially complete (e.g., 10% or more, 25% or more, 50% or more, or 75% or more), substantially complete (e.g., 85% or more, 90% or more, or 95% or more), or fully complete (e.g., 98% or more, or 99% or more). Gene silencing may be accomplished using a variety of methods known in the art. In some embodiments, silencing is performed using gene editing. The terms “gene editing,” “genome editing,” or “genome engineering,” may be used interchangeably herein to refer to a type of genetic engineering in which DNA is inserted, deleted, modified, or replaced in the genome of a living organism. For example, gene editing may be used to disrupt or modify an endogenous genomic region of a host cell, inserting an exogenous gene into a host genome, replacing an endogenous nucleotide sequence with an exogenous nucleotide sequence, or any combination thereof. Systems and methods for gene editing are described in detail in, e.g., National Academies of Sciences, Engineering, and Medicine; National Academy of Medicine; National Academy of Sciences; Committee on Human Gene Editing: Scientific, Medical, and Ethical Considerations. Human Genome Editing: Science, Ethics, and Governance. Washington (DC): National Academies Press (US); 2017 Feb. 14. A, The Basic Science of Genome Editing.

In some embodiments, gene editing is performed using a CRISPR/Cas system or method. As used herein, the term “CRISPR/Cas system” refers collectively to transcripts and other elements involved in the expression of and/or directing the activity of CRISPR-associated (“Cas”) genes, including sequences encoding a Cas gene, Cas protein, a tracr (trans-activating CRISPR) sequence (e.g., tracrRNA or an active partial tracrRNA), a cr (CRISPR) sequence (e.g., crRNA or an active partial crRNA), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Staphylococcus aureus or Streptococcus pyogenes. In certain embodiments, the Cas9 protein can be included in the system separate from, associated with, or encoded by, a vector.

Any element of any suitable CRISPR/Cas gene editing system known in the art can be employed in the systems and methods described herein, as appropriate. CRISPR/Cas gene editing technology is described in detail in, for example, Cong et al., supra; Xie et al., supra; U.S. Patent Application Publication 2014/0068797; U.S. Pat. Nos. 8,697,359; 8,771,945; and 8,945,839; US2010/0076057; US2011/0189776; US2011/0223638; US2013/0130248; WO/2008/108989; WO/2010/054108; WO/2012/164565; WO/2013/098244; WO/2013/176772; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; and US20140170753; Makarova et al., Nature Reviews Microbiology, 9(6): 467-477 (2011); Wiedenheft et al., Nature, 482: 331-338 (2012); Gasiunas et al., Proceedings of the National Academy of Sciences USA, 109(39): E2579-E2586 (2012); Jinek et al., Science, 337: 816-821 (2012); Carroll, Molecular Therapy, 20(9): 1658-1660 (2012); Al-Attar et al., Biol Chem., 392(4): 277-289 (2011); and Hale et al., Molecular Cell, 45(3): 292-302 (2012).

The one or more genes that are downregulated in cancer associated fibroblasts may be any such gene known in the art, or any gene identified by the methods described in the Examples below. In some embodiments, the one or more genes that are downregulated in CAFs are one or more extracellular matrix crosslinking genes, such as the HAPLN1 gene, the genipin gene, and/or the tissue transglutaminase (TG2) gene. Genipin is a gardenia fruit extracted collagen crosslinker and has potential to attenuate cancer proliferation. Transglutaminase is an evolutionary conserved enzyme and contributes significantly to the organization of ECM by mediating cell-matrix interactions that affect cell spreading and migration, and is crucial for wound healing. HAPLN1 stabilizes the aggregates of proteoglycan monomers with hyaluronic acid in the ECM, and HAPLN1 can suppress melanoma metastasis by ECM stabilization.

The one or more genes that are upregulated in carcinoma-associated fibroblasts may be any such gene known in the art, or any gene identified by the methods described in the Examples below. Soluble factors such as TGF-β known to be secreted by cancer cells can induce CAFs via TGF-β and STAT3 signaling pathways. Thus, in some embodiments, the one or more genes upregulated in CAFs may be any gene that is involved in TGF-β and/or STAT3 signaling pathways, such as the TGFBR1 and/or STAT3 genes.

The composition described herein may comprise a pharmaceutically acceptable carrier. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The disclosure also provides a method of inhibiting extracellular matrix (ECM) remodeling in the microenvironment of a cancerous tumor, which comprises administering the above-described composition to the microenvironment of a cancerous tumor, whereupon one more genes downregulated in CAFs are expressed and/or one or more genes upregulated in cancer CAFs are silenced in the microenvironment of the cancerous tumor, thereby inhibiting ECM remodeling in the microenvironment of the cancerous tumor. In other embodiments, the disclosure provides a method of inhibiting extracellular matrix (ECM) remodeling in the microenvironment of a cancerous tumor comprising administering to the microenvironment of a cancerous tumor a composition comprising one or more extracellular matrix crosslinking proteins and a pharmaceutically acceptable carrier, whereby ECM remodeling in the microenvironment of the cancerous tumor is inhibited. The disclosure also provides a method of inhibiting progression of a cancerous tumor, which method comprises administering the above-described composition to the microenvironment of a cancerous tumor, whereupon one more genes downregulated in CAFs are expressed and/or one or more genes upregulated in cancer CAFs are silenced in the microenvironment of the cancerous tumor, thereby inhibiting progression of the cancerous tumor.

The term “extracellular matrix (ECM),” as used herein, refers to the non-cellular component present within all tissues and organs, which provides not only essential physical scaffolding for the cellular constituents but also initiates biochemical and biomechanical cues that are required for tissue morphogenesis, differentiation, and homeostasis. The ECM comprises a three-dimensional network of extracellular macromolecules, such as collagen, enzymes, and glycoproteins. ECM remodeling in the tumor microenvironment (TME) by colorectal cancer (CRC), breast cancer, and melanoma cells is known to be associated with tumor progression, and CAFs have been shown to remodel the tumor microenvironment to promote efficient metastasis.

The term “tumor,” as used herein, refers to an abnormal mass of tissue that results when cells divide more than they should or do not die when they should. In the context of the present disclosure, the term tumor may refer to tumor cells and tumor-associated stromal cells or tissue (i.e., the tumor “microenvironment”). Tumors may be benign and non-cancerous if they do not invade nearby tissue or spread to other parts of the organism. In contrast, the terms “cancerous tumor,” “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The cancerous tumor may be a carcinoma (cancer arising from epithelial cells), a sarcoma (cancer arising from bone and soft tissues), a lymphoma (cancer arising from lymphocytes), a blood cancer (e.g., myeloma or leukemia), a melanoma, or brain and spinal cord tumors. The cancerous tumor can be located in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. More particularly, cancers of the digestive system can affect the esophagus, stomach, small intestine, colon, rectum, anus, liver, gall bladder, and pancreas. Cancers of the respiratory system can affect the larynx, lung, and bronchus and include, for example, non-small cell lung carcinoma. Cancers of the reproductive system can affect the uterine cervix, uterine corpus, ovaries, vulva, vagina, prostate, testis, and penis. Cancers of the urinary system can affect the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). In one embodiment, the cancerous tumor is a colorectal cancer or carcinoma (CRC), a breast cancer, or a melanoma.

The disclosure also provides a method of inhibiting progression of a cancerous tumor, which method comprises: (a) genetically engineering a population of fibroblasts obtained from a subject to (a) express one or more genes that are downregulated in cancer associated fibroblasts (CAFs) and/or (b) silence one or more genes that are upregulated in CAFs; (b) administering the genetically engineered population of fibroblasts to a cancerous tumor tissue present in the subject, whereupon the one more genes downregulated in CAFs are expressed and/or the one or more genes upregulated in cancer CAFs are silenced in the cancerous tumor tissue, thereby inhibiting progression of the cancerous tumor in the subject. Descriptions of CAFs, genes upregulated or downregulated in CAFs, and components thereof set forth above also apply to those same aspects of the aforementioned method of inhibiting progression of a cancerous tumor.

The compositions or population of fibroblasts described herein can be administered to the microenvironment of a cancerous tumor (e.g., a cancerous tumor in a human subject) using standard administration techniques, including intratumoral, oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered using peripheral systemic delivery by intravenous, intraperitoneal, or subcutaneous injection.

In some embodiments, compositions or populations of fibroblasts described herein may be targeted to specific tumor sites or tumor cell populations. Tumor-specific drug targeting methods are known in the art and may be used in connection with the present disclosure. Such targeting methods include, for example, (i) passive targeting based on the specific features of tumor vasculature, (ii) active targeting with specific binding of an antitumor agent (e.g., the compositions or engineered fibroblasts described herein) with its molecular target, and (iii) cell-mediated tumor targeting. It will be appreciated that passive targeting is associated with the structural features of the tumor vasculature, while active targeting typically involves covalent or non-covalent binding of an antitumor agent (e.g., the compositions or engineered fibroblasts described herein) to a molecule which is capable of selective interaction with specific molecules on the surface of target cells. Cell-mediated tumor targeting involves drug delivery by cells which possess preferential tropism to a tumor type. Tumor-specific drug targeting methods and agents are further described in, e.g., Kutova et al., Cancers, 11, 68 (2019); doi:10.3390/cancers11010068.

Ideally, the methods described above result in the treatment of the cancerous tumor. As used herein, the terms “treatment” and “treating” can include reversing, alleviating, inhibiting the progression of, preventing or reducing the likelihood of a cancer, or one or more symptoms or manifestations of a cancer. Preventing refers to causing a cancer, or symptom or manifestation of such, or worsening of the severity of such, not to occur. Accordingly, the disclosed compositions can be administered prophylactically to prevent or reduce the incidence or recurrence of a cancer. In some embodiments, the disclosed methods promote inhibition of tumor cell proliferation, progression, the eradication of tumor cells, and/or a reduction in the size of at least one tumor such that a mammal (e.g., a human) is treated for cancer. By “treatment of cancer” is meant alleviation of cancer in whole or in part. In one embodiment, the disclosed method reduces the size of a cancerous tumor by at least about 20% (e.g., at least about 25%, 30%, 35%, 40%, 45%, 50%, 55%, 60%, 65%, 70%, 75%, 80%, 85%, 90%, or 95%). Ideally, the cancerous tumor is completely eliminated.

The methods may be performed ex vivo or in vivo. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. When the cell is contacted with the composition in vivo, the composition may be administered to an animal, such as a mammal, particularly a human, using standard administration techniques and routes, such as those described herein.

When a population of fibroblasts, or a composition comprising fibroblasts, is administered to a subject (e.g., a human), the cells can be allogeneic or autologous to the subject. In “autologous” administration methods, cells (e.g., fibroblasts) are removed from a subject, stored (and optionally modified), and returned back to the same mammal. In “allogeneic” administration methods, a subject receives cells (e.g., fibroblasts) from a genetically similar, but not identical, donor. Preferably, the cells are autologous to the subject.

The “subject” treated by the disclosed methods is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals such as, but not limited to, primates (e.g., humans, monkeys, apes, etc.); bovines (e.g., cattle, oxen, etc.); ovines (e.g., sheep); caprines (e.g., goats); porcines (e.g., pigs, hogs, etc.; equines (e.g., horses, donkeys, zebras, etc.); felines, including wild and domestic cats; canines, including dogs; lagomorphs (e.g., rabbits, hares, etc.); and rodents (e.g., mice, rats, etc.). An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Materials and Methods

The following materials and methods were used in the experiments described in the Examples.

Cell culture and transfection. EpH4-Ev and 4T1 cells were acquired from ATCC. 67NR cells were acquired from Karmanos Cancer Institute. GFP-transfected NIH-3T3 cells were acquired from Cell Biolabs. All the cell lines were cultured in Dulbecco's Modified Eagle Medium (DMEM) (Thermo Fisher) supplemented with 10% FBS (Thermo Fisher) and 1% penicillin-streptomycin (Thermo Fisher) at 37° C. in 5% CO2. RhoA^{T19N} transfection was performed using lipofectamine 3000 (Thermo Fisher). RhoA^{T19N} plasmid was a gift from Dr. Gary Bokoch (Addgene plasmid #12967; http://n2t.net/addgene:12967; RRID: Addgene_12967).

Coculture. Spheroid formation: the hanging drop method and hydrophobic wells were used sequentially to form spheroids: Cells were resuspended in DMEM at the density of 5×10⁴ cells per 40 μL and placed on the inner side of the lid of a cell culture dish. The droplets were placed at the interval of 300 mm. The dish was filled with 10 mL of PBS to provide humidity for the spheroids. The lid was then replaced back on to the dish. After incubation for 2 days, the spheroids were transferred to an ultra-low attachment 96-well plate (Corning, CLS7007) prefilled with 100 μL DMEM. The spheroids were then harvested for experiments within 4 days.

Spheroid placement: rat tail type I collagen (Corning, 354236) was diluted to 3 mg/mL by a mixture of 10×DMEM (Sigma-Aldrich, D2429) and 0.1 M NaOH at 3:1 ratio. Prior to embedding the spheroid, wells in a 24-well glass bottom dish (Cellvis, P24-0-N) were coated with 500 μL of 0.1% (w/v) Poly-L-Lysine solution (Sigma-Aldrich, P8920) for overnight at 4° C. Upon the removal of Poly-L-Lysine solution, the wells were washed with 500 μL of PBS for 10 minutes, followed by air drying. 15 μL of collagen was added to the center of the well first and allowed to reach a partial gelling state at room temperature for 15 minutes. Then the spheroid was transferred from the ultra-low attachment well to the center of the glass bottom well, where the spheroid was allowed to adhere to the gelling collagen for 30 minutes.

Spheroid encapsulation without fibroblast: after the spheroid adhered to the glass bottom well via collagen, the liquid collagen kept at the 4° C. was poured to the well to embed the spheroid. 40 μL liquid collagen was slowly pipetted into the well and allowed to gel for 2 hours at room temperature. 2 mL DMEM was then added into the well. The embedded spheroid was incubated for at least 1 day or longer before being used.

Spheroid encapsulation with fibroblasts: for coculture, 40 μL collagen was used to re-suspend 2×10⁵ NIH-3T3 cells after centrifugation. The fibroblasts-containing liquid collagen was then added to the glass bottom well where a spheroid was placed in its center. The mixture was allowed to gel at room temperature for 2 hours. 2 mL DMEM was then added into the well. The embedded spheroid was incubated for at least 1 day or longer, as indicated in the text, before being used.

ECM alignment by an external magnetic field. The liquid collagen (3 mg/ml) was used to re-suspend fibroblasts to reach density of 10⁶ cells/mL. The fibroblasts-containing collagen was then mixed with 200-nm magnetic particles (Chemicell, screenMAG/RR-Protein G, 2 mg/mL) in a ratio of 3:1. 10 μL of the mixture was added to a flow chamber (Ibidi, μ-Slide VI 0.1) and treated with a pair of magnets positioned at both sides at room temperature for 10 minutes to align the fibrils.

For the control group, the mixture was placed at room temperature for 10 minutes without the magnets, followed by adding 50 μL medium in the both reservoirs of flow chamber and cultured in the incubator for 2 days. Afterwards, supernatant harvested from 67NR-cultured medium was added to one of the reservoirs, with the other filled with regular medium. Upon two days of culturing, cells in the flow channel were fixed with 4% paraformaldehyde for immunofluorescence.

ECM crosslinking. ECM-encapsulated 67NR spheroids were prepared as previously described. After 1-hour incubation, genipin (Sigma-Aldrich, G4796) was mixed with DMEM to reach final concertation of 0.5 mM and 1 mM. The solutions were then added to the samples and incubated for 24 hours. The DMSO-added DMEM were used as a control for the experiment. The samples were then washed with PBS for further usage.

Elastic modulus measurement. The Large Angle Oscillatory Shear (LAOS) was used to test the shear modulus of the crosslinked and uncrosslinked collagen gel. The collagen gel was prepared in disks and subjected to sinusoidal rotational deformation. The amplitude of the applied strain (y) to the material was increased at a fixed rotational frequency (w) of 1 rad/s. The analysis utilized a viscoelastic stress response model wherein the shear stress is computed as a function of strain ranging from 0.01%-15%. The Fourier Transform method was utilized to quantify the nonlinear stress response of ECM samples under increasingly large angular shear strain. LAOS was performed using an Anton Paar Modular Compact Rheometer (MCR 302) with a parallel plate (diameter 8 mm) at 37° C. The measurement values of the shear modulus were then used obtain the elastic modulus by the formula:

E=2G(1+v)

where E is elastic modulus, G is shear modulus, and v is the Poisson ratio. The Poisson ratio was assumed to be 0.5.

Immunofluorescence. Samples were fixed in 4% paraformaldehyde for 30 minutes, permeabilized with 0.1% Triton X100 (Sigma-Aldrich, X100) in PBS for 30 minutes. PBS containing 2% bovine serum albumin (Sigma-Aldrich, A7906) and 0.1% Tween-20 (Promega) was then added to sample for 30 minutes for blocking. All antibody was diluted in PBS with 1% bovine serum albumin. The fixed cells were incubated with the primary antibody for overnight at 4° C., followed by washing using PBS for 3 times, and then incubated with the secondary antibody for 2 hours. After washing, the samples were immersed in PBS and stored in 4° C. for further usage. Dilution of antibodies used as follows: rabbit anti-FSP1 antibody (Millipore, S100A4, 1:500 dilution), mouse anti-αSMA antibody (Thermo Fisher, 1A4, 1:500 dilution), goat anti-rabbit IgG antibody conjugated with Alexa 647 (Jackson ImmunoResearch, 1:500 dilution), goat anti-mouse IgG antibody conjugated with DyLight 594 (abcam, 96873, 1:2000 dilution), goat anti-mouse IgG Antibody conjugated with Alexa 647 (BioLegend, 405322, 1:250 dilution). To avoid the strong autofluorescence emitted from crosslinked ECM as the result of genipin reacting with amino acids in the red channel, the immunostaining of the two CAF markers were performed separately in separate samples, both using the secondary antibody conjugated with Alexa 647.

Confocal microscopy. Imaging was performed using Leica TCS SP8 confocal microscope. Live cell imaging was performed with a 63× objective (NA 1.4) with pinhole set at one airy unit. An incubation chamber was used to maintain 37° C., 5% CO2, and humid air. Brightfield images were acquired in the transmitted light mode. 655-nm was used for IRM to visualize ECM fibrils. All the images of immunofluorescence were acquired by a 40× objective (NA 1.4) with pinhole was set at two airy units.

Exosome depletion. To remove debris, medium harvested from the 67NR culture was first centrifuged at 1,000 rpm for 10 minutes. The supernatant was transferred to a 4 mL ultracentrifuge tube (Beckman ULTRA-CLEAR™) and centrifuged in a SW 60 Ti rotor (Beckman) at 40,000 rpm and 4° C. for 2 hours. The supernatant was then collected and used to culture fibroblasts. In addition, the harvested medium not subjected to ultracentrifugation was used as the control.

Traction force microscopy. The Silicone substrates (CY 52-276 A:B=1:1) (Dow Corning) were prepared as previously described at room temperature. The elastics modulus of the substrate was ˜3 kPa. To conjugate microbeads fiduciary on the substrate, rhodamine carboxylate-modified microbeads (Thermo Fisher, F8801) were diluted from the tock in PBA at the ratio 1:25000. The bead solution was mixed with EDC (1-ethyl-3-(3-dimethylaminopropyl) carbodiimide hydrochloride (Thermo Fisher, 22980) to achieve the final concentration of 200 μg/mL. The Silicone substrates were treated with 2% APTS ((3-Aminopropyl) triethoxysilane) (Sigma-Aldrich, 440140) diluted in PBS for 5 minutes at room temperature before the EDC-treated bead solution was added to the surface. The mixture was set to react for 4 hours at room temperature. Afterwards, to minimize cytotoxicity, the substrates were immersed in PBS for 1 hour at room temperature. Before placing spheroids on the substrate, 200 μg/mL rat tail Type I Collagen was used to coat the surface for 1 hour at 37° C. Spheroids were then placed following the steps similar to the steps described above. Images documenting the positions of the fiduciary microbeads were first acquired with the spheroids adhered firmly to the substrate. The trypsinization (5%) was performed on-stage to detach the spheroid, followed by imaging the fiduciary microbeads again. The images before and after trypsinization were then analyzed using the Traction Force Microscopy plugin for ImageJ45.

Fluorescence recovery after photobleaching. 10 mg/mL of 2,000 kDa fluorescein isothiocyanate (FITC)-Dextran (SigmaAldrich, FD2000S) was added to the collagen gel and incubated for 30 minutes at 37° C. The dimensions used for photobleaching were 60 μm×10 μm, 60 μm×5 μm, and 20 μm×2 μm for samples containing spheroids only, magnetically aligned ECM, and genipin-treated ECM, respectively. For the analysis, photobleaching was corrected using an exponential fit:

A×e^{tB}

where t is the time point and A and B are fitting coefficients. Then on the bleaching corrected recovery curve, a fitting equation of the following form was employed:

a×(1−e^{tB})+c

where t is the time point and a, b and c are fitting coefficients to estimate the recovery time. The analysis was performed for both curves obtained through the radial and the orthogonal stripes. The characteristic fluorescence recovery times τ_{D}, when 64% of the fluorescence intensity is recovered, was calculated based on the fitted curve.

Imaging analysis. The cell velocity was measured using the free software CellTracker. To measure the migration directionality, the line between the centroid of the cell and the centroid of line was first drawn, and the line linked the centroids of the same cell in the first and last frames was drawn. The angle between the two lines was then measured.

Fibril coherence analysis was performed using Quantitative orientation measurement in orientationJ21. The immunostaining was quantified using Fiji/ImageJ software. The noise from the background were subtracted by the following formula:

$$\frac{{I}_{2} \times {A}_{2} - {I}_{1} \times {A}_{1}}{{A}_{2} - {A}_{1}}$$

where I_{I} and A_{I} denotes the fluorescence intensity, and the area of the cell, respectively. I₂ and A₂ denotes the fluorescence intensity, and the area of the larger region encompassing the cell and the surrounding background, respectively. The larger region was traced by hand. The corrected fluorescence intensity was then recorded for each cell.

Statistical analysis. The box and whisker plots were produced by the software GraphPad Prism. The box ranges from 25-75th percentile, with the middle line indicating the median, and the whisker indicating the minimum and maximum. The P values were calculated by two-tailed unpaired Student's t-test.

### Example 1

This example describes the coculture of early-stage breast cancer cells and fibroblasts and shows ECM fibrils alignment and fibroblast activation.

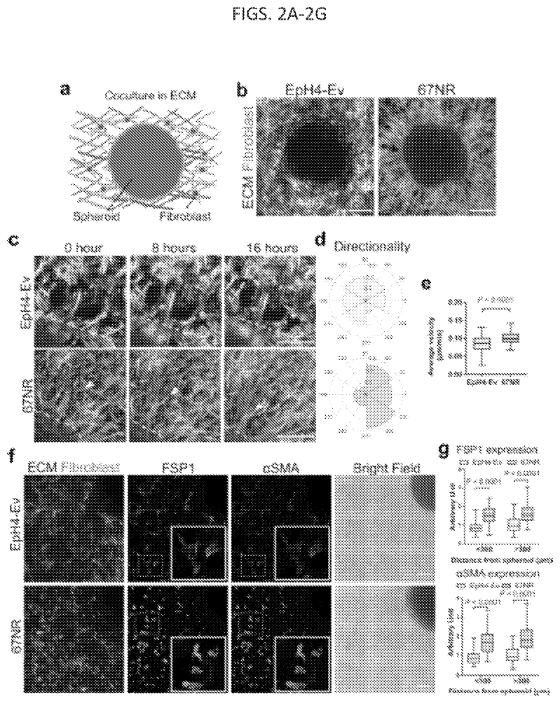

To examine the interaction between cancer cells and the fibroblasts in the stroma, spheroids consisting of mouse mammary epithelial cells EpH4-Ev, 67NR, or 4T1 were embedded in the 3D ECM, where fibroblasts NIH-3T3 were also present, mimetic of the distribution of these two cell types in the mammary gland (FIG. 2A). EpH4-Ev, 67NR and 4T1 cells were chosen because they respectively exhibit characteristics typical of normal, non-metastatic cancer and metastatic cancer cells from the same mouse strain. To distinguish between cell types in cocultures, fibroblasts were permanently transfected with GFP. ECM fibrils were imaged using internal reflectance microscopy (IRM). After 24-hour of coculturing, ECM fibrils in 67NR-fibroblast cocultures were radially aligned, whereas fibrils in the EpH4-Ev-fibroblast coculture remained isotropically oriented (FIGS. 2B, 2C). Notably, fibroblasts cocultured with the 67NR spheroids showed directionally persistent migration toward the spheroid, whereas the fibroblasts with EpH4-Ev migrated without preferences (FIG. 2D). The average velocity of fibroblasts cocultured with 67NR was 1.2-fold higher than the ones with EpH4-Ev (FIG. 2E). To examine whether cell migration pattern is associated with CAF induction, the cocultured fibroblasts were examined by immunofluorescence against widely used CAF markers FSP1 and αSMA. The fibroblasts cocultured with 67NR spheroids showed 1.8-fold and 2.0-fold higher FSP1 and αSMA expression, respectively, than the ones with EpH4-Ev spheroids, when located within the 300-μm perimeter from the spheroid (FIGS. 2F-2G). Additionally, 1.6-fold and 1.9-fold higher FSP1 and αSMA expression than the control, respectively, were observed in the fibroblasts 300-μm away from the 67NR spheroid (FIG. 2G), suggesting CAFs are induced by long-range mechanisms. As 4T1 spheroids rapidly disintegrated, a characteristic attributed to its high metastatic potential, 4T1 cells were not used for the following experiments.

### Example 2

This example demonstrates early-stage breast cancer cells align the ECM fibrils and facilitate faster diffusion towards stroma.

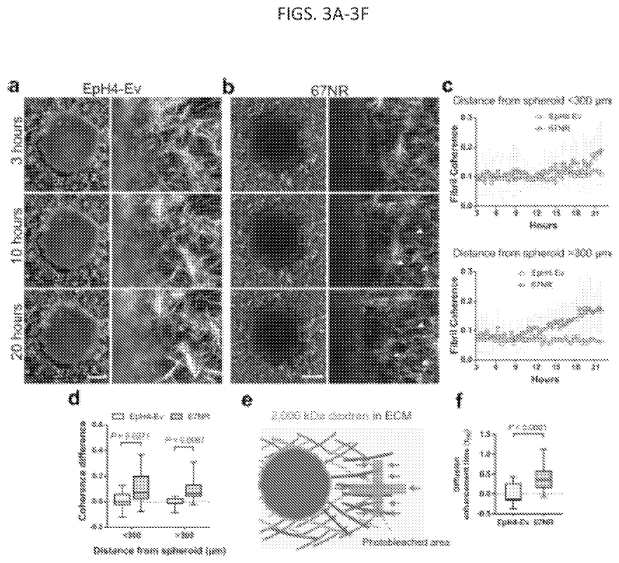

The observation that fibrils were radially aligned in 67NR-fibroblast coculture, but not in EpH4-Ev-fibroblast coculture, suggests 67NR is important for alignment. To determine whether 67NR spheroid alone is sufficient to align the fibrils, time-lapse imaging was performed by embedding EpH4-Ev and 67NR spheroids in ECM without fibroblasts. After 24-hour incubation, the EpH4-Ev spheroid-embedded fibrils remained isotropic (FIG. 3A), whereas 67NR spheroid-embedded fibrils were aligned (FIG. 3B). To quantify the degree of fibril alignment, coherence analysis 21 was performed. Higher coherence scores represent more alignment. Fibril orientations within and outside the 300-μm perimeter from the spheroid were evaluated separately. The score associated with EpH4-Ev spheroids remained unchanged, whereas the score associated with 67NR spheroids gradually increased (FIGS. 3C-3D). The coherence score increased by 0.1 from 3-hour to 20-hour incubation for fibrils incubated with 67NR, whereas the score with EpH4-Ev remained unchanged (FIG. 3D).

It was postulated that there is a causal relationship between the simultaneous observations of radially aligned fibrils in 67NR-fibroblast coculture and higher CAF marker expression, possibly mediated by exosomes. Exosomes secreted by cancer cells can activate fibroblast via TGF-β and miRNA contained within. CAF phenotypes might be induced by exosomes secreted from the 67NR spheroid; and the fibril alignment favored efficient exosome transport in ECM. First, it was verified whether exosomes secreted by 67NR are responsible for the increase expression of CAF markers in fibroblasts: the medium from 67NR culture was collected and removed exosomes by ultracentrifugation. Fibroblasts cultured in the exosome-depleted supernatant showed ˜20% less FSP1 and αSMA expression than the ones cultured in the uncentrifuged medium, suggesting the observed CAF marker upregulation in fibroblasts resulted from the exosomes secreted by 67NR.

Next, it was examined whether the fibril alignment can affect exosome diffusion. Line fluorescence recovery was performed after photobleaching (FRAP) to measure the diffusivity of the spheroid-embedded ECM. The 2,000-kDa FITC-conjugated dextran was used as the bleachable probe added to the ECM, because its molecular size is comparable to the exosomes secreted by 67NR25. Two orthogonally oriented stripes were selected as the region of interest for photobleaching: one in the radial direction to spheroid, the other perpendicular to the first stripe (FIG. 3E). To assess whether there is enhancement in diffusion rates in the radial direction from the spheroid, the metric “diffusion enhancement time (τ_{DE})” was introduced, defined as the difference in the characteristic fluorescence recovery times τ_{D}, when 64% of the fluorescence intensity is recovered, between the two stripes:

r_{DE}=r_{D(radial)}−r_{D(orthogonal)}

where r_{D(radial)} represents the r_{D} measured in the radial direction, and r_{D(orthogonal)} in the corresponding orthogonal direction. If the diffusion rate is faster in the radial direction, r_{D(orthogonal)} will be shorter, since the orthogonal stripe presents a smaller photobleached gap for the radially diffusing FITC-dextran to fill (FIG. 3E). Therefore, positive r_{DE} values represent faster diffusion rates in the radial direction compared to the orthogonal direction. The r_{DE} value in 67NR spheroid-embedded ECM was 0.4 second, and approximately zero for EpH4-Ev-embedded ECM. Taken together, the results suggest radially aligned fibrils facilitate faster radial diffusion of exosomes (FIG. 3F).

### Example 3

This example demonstrates that fibril alignment enhances exosome diffusion and CAF induction.

To test whether the fibril alignment and the subsequent enhanced exosome diffusion induce CAF phenotypes, collagen mixed with 200-nm paramagnetic particles and fibroblasts was placed in a uniform magnetic field and allowed to polymerize.

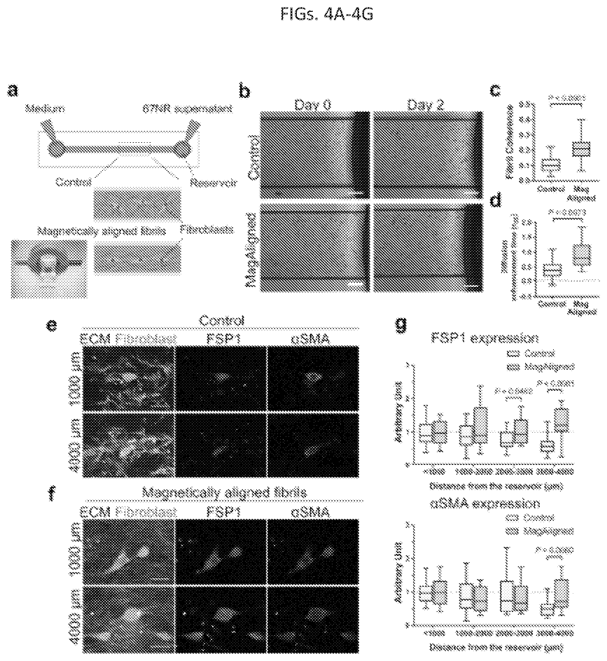

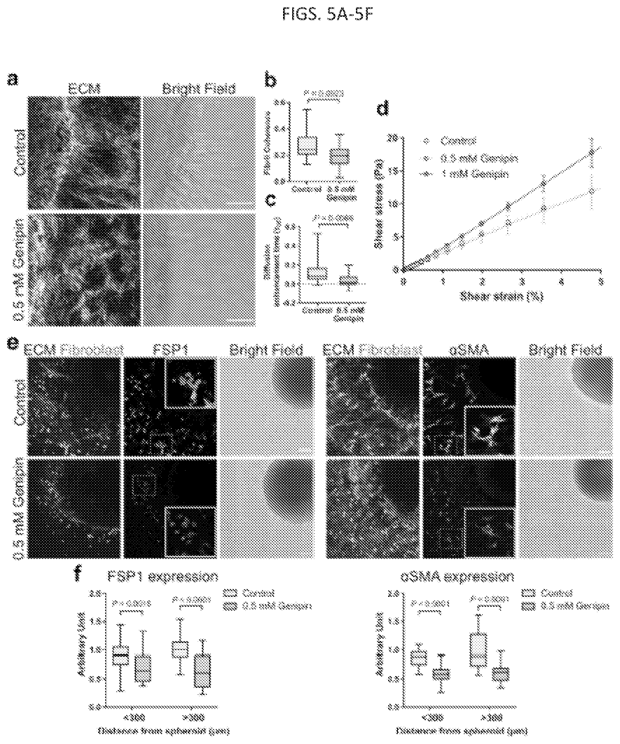

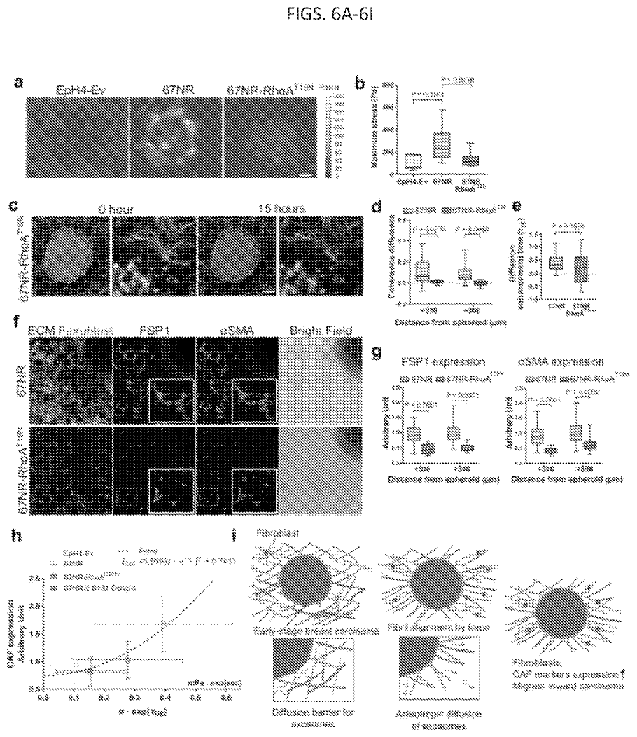

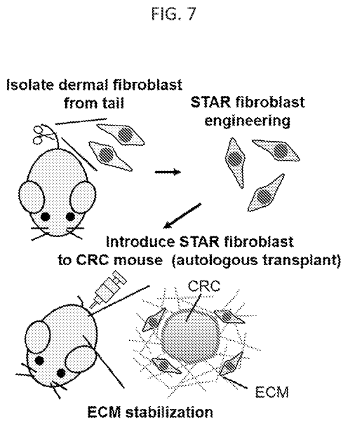

Upon polymerization, medium harvested from 67NR culture was supplied through a reservoir (FIG. 4A). Visually, ECM fibrils polymerized in the magnetic field were aligned, whereas the control remained isotropic (FIG. 4B). The coherence score of fibrils subjected to the magnetic field was higher by 0.1 than the control (FIG. 4C). The line FRAP results correspondingly showed 2.1-fold higher r_{DE} values in ECM subjected to magnetic field than the control (FIG. 4D). These results suggest that the magnet field effectively aligned fibrils and enhanced directional diffusion. Immunostaining was then performed to evaluate CAF induction. In the unaligned group, fibroblasts located more than 3000-μm away from the reservoir showed 39% and 47% lower FSP1 and αSMA expression, respectively, than the ones located within 1000-m. In contrast, there was no difference between the two locations in the aligned group (FIGS. 4E-4G). These results led us to deduce that fibril alignment enhances the anisotropic diffusion of exosomes and facilitates them to reach the fibroblasts further away from the cancer cells.

### Example 4

This example demonstrates that prevention of ECM reorganization attenuates CAF induction.

The corollary to the observation that ECM fibril alignment promotes CAF induction in the coculture is that suppressing such alignment will attenuate it. To confirm this, genipin, a biocompatible crosslinking reagent, was used to suppress the fibril reorganization. The optimal genipin concentration was identified to be 0.5 mM. 67NR-embedded ECM treated by 0.5 mM genipin for 24 hours exhibited no observable cytotoxicity and negligible ECM stiffening (FIG. 5d). Yet the fibril alignment was suppressed by 28% (FIGS. 5A-5B). Correspondingly, the r_{DE} value decreased to a negligible level (FIG. 5C). Though ECM crosslinking might lead to CAF induction because of ECM stiffening, it was not the case in these experiments. The rheological measurement results showed that the elastic modulus of the ECM treated with 0.5 mM genipin was 755±163 Pa (mean±s.d.), comparable to the un-crosslinked ECM, while ECM treated with 1 mM genipin became stiffer by 29% (FIG. 5D). Having had established the optimal genipin concentration to suppress fibril alignment, it was used to evaluate whether CAF induction in cocultured fibroblasts would be attenuated. As expected, in genipin-treated cocultures, the FSP1 and αSMA expression in the fibroblasts was reduced by 23% and 33% respectively within the 300-μm perimeter from the spheroid. The FSP1 and αSMA expression was further decreased by 36% and 39% respectively, when comparing fibroblasts located outside 300-μm perimeter between the treatment and the control (FIGS. 5E-5F). To exclude the possibility that genipin directly downregulates CAF markers in fibroblasts, their expression was evaluated in genipin-treated samples containing fibroblasts only. The FSP1 and αSMA expression remained unchanged in the fibroblast-only samples after genipin treatment, suggesting genipin reduces CAF marker expression in fibroblasts not in a direct manner, but through the suppression of ECM fibril alignment.

### Example 5

This example demonstrates that fibril alignment and subsequent CAF induction by cancer cells is force-dependent.

Reorientation of fibrils was prominent in the time-lapse images when 67NR spheroids were present, implying that fibril alignment is a mechanical process and force-dependent. To verify this, 2D traction force microscopy was performed to compare forces exerted by 67NR and EpH4-Ev spheroids, the spheroid-containing ECM was polymerized above a thin PDMS film decorated with fiducial fluorescent nanoparticles, so that forces generated by the spheroid displaced the nanoparticles, thereby informing force magnitudes generated by the spheroid. Adopting the assumption in previous studies that force generation by spheroids is isotropic, and 2D results are proportional to 3D values, this measurement sufficed to compare the relative force generation capacity between 67NR and EpH4-Ev spheroids. 67NR spheroids generated 2.5-fold higher traction forces than EpH4-Ev (FIGS. 6A-6B). This higher force generation correlated with the more aligned ECM fibrils. To further prove the causality between forces, fibril alignment and CAF induction, 67NR cells expressing dominant negative form of RhoA (RhoA^{T19N}) were used in the coculture, so that RhoA-mediated force generation was inhibited. The traction force microscopy results verified that 67NR-RhoA^{T19N} spheroids generated 51% less forces than their wildtype counterparts (FIGS. 6A-6B). 67NR-RhoA^{T19N} spheroids showed negligible radial alignment (FIGS. 6C-6D). Furthermore, r_{DE} in the 67NR-RhoA^{T19N} spheroid-embedded ECM was 59% less than r_{DE} in the ECM with wildtype 67NR spheroids (FIG. 6E). Taken together, the results agree with the model where the early-stage breast cancer cells use high forces to align ECM fibrils and enhance exosome diffusion in the radial direction.

It was next investigated whether suppressing fibril alignment would reduce CAF induction in 67NR-RhoA^{T19N}-fibroblast cocultures. In terms of migration, it was observed that the fibroblasts cocultured with 67NR-RhoA^{T19N} spheroids migrated without preferences, with the average velocity 29% less than the ones with wildtype 67NR spheroids. The FSP1 and αSMA expression was attenuated by 51% and 54%, respectively, in fibroblasts within the 300-μm perimeter from the 67NR-RhoA^{T19N} spheroid; and decreased by 51% and 36%, for the ones outside the perimeter (FIGS. 6F, 6G). To exclude the possibility that the decreased CAF induction directly resulted from impaired exosome secretion in 67NR-RhoA^{T19N} cells, media was harvested from 67NR-RhoA^{T19N} and wildtype 67NR cultures to incubate fibroblasts for 2 days. Fibroblasts incubated with media from 67NR-RhoA^{T19N} and wildtype 67NR cultures expressed αSMA and FSP1 at comparable levels, indicating the secretion of CAF-promoting factors is not impaired in 67NR-RhoA^{T19N} cells. Overall, the results demonstrate that force-dependent fibril alignment contributes to enhancing diffusion of CAF-promoting exosomes and subsequent CAF induction by early-stage breast cancer cells.

Additionally, high forces generated by cancer cells can potentially induce CAFs through other synergetic effects: it is plausible that as the ECM stiffens, resulting from force-dependent ECM fibril alignment, CAF phenotypes are reinforced in the fibroblasts; high forces might further activate mechanosignaling pathways, also contributing to CAF induction. Based on these data, the relation

I_{CAF}=α(σ·e^{r}^{_{DE}})²I_{baseline}

could be used to gain insights as to whether and to which extent forces contribute to CAF induction independent of enhanced diffusion (FIG. 5H), where I_{CAF} represents the index of CAF marker expression, a is the maximum traction stress generated by the spheroid, r_{DE} the diffusion enhancement time, I_{baseline} is the baseline expression of CAF markers in non-CAF cells, and a is a coefficient which can be derived from curve fitting. In this study, a value was determined to be 0.7451 and I_{baseline} was determined to be 0.4249.

In summary, the above results show that early-stage cancer cells can generate high forces to reorient ECM fibrils in the tumor microenvironment. Such reorientation results in radially aligned ECM fibrils where exosomes secreted by cancer cells diffuse more efficiently to reach fibroblasts in the periphery. The CAF-promoting factors in the exosomes then upregulate genes manifesting CAF phenotypes in the fibroblasts (FIG. 6I). By either inhibiting force generation or directly suppressing ECM fibril alignment, CAF induction by cancer cells can be reversed.

### Example 6

This example describes comparative proteomic studies between dermal fibroblasts, colonic fibroblasts and CAFs.

Three desired features should be exhibited by “STabilization Against Remodeling (STAR)” fibroblasts: minimal immunogenicity, resistance to CAF induction, and capacity to suppress ECM remodeling and repair damages by cancer cells and CAFs. To minimize the immune responses to cell therapy, dermal fibroblasts from the same mouse with CRC will be collected in the tail (FIG. 7) as the source of STAR fibroblasts. In order to establish STAR fibroblasts capable of resisting CAF induction and suppressing ECM remodeling, the differences in the gene expression between normal fibroblasts and CAFs will first be studied. Though it is known soluble factors such as TGF-β secreted by cancer cells induces CAFs, via TGF-β and STAT3 signaling pathways, a comprehensive profiling might further reveal additional pathways involving in CAF induction. Those pathways should also be downregulated. Similarly, though secreting appropriate amount of ECM crosslinking enzymes can potentially suppress tumor progression, additional secreted molecules promoting tumor suppression might be discovered by mass spectrometry. Those genes encoding for the additional secreted molecules should be upregulated in STAR fibroblasts. To identify the gene whose expression requires genomic editing in STAR fibroblasts, primary colonic fibroblasts in the normal or tumor stroma will be collected for the mass spectrometry, alongside with dermal fibroblasts. Fibroblasts in the colonic tissues from control, vehicle-treated and AOM/DSS-treated mice will be isolated and cultured by previously established techniques. The primary fibroblasts can be steadily maintained for 6 passages before becoming senescent. To characterize the difference between the normal dermal fibroblasts, normal colonic fibroblasts and CAFs, exploratory mass spectrometry will be performed. Lysates of dermal fibroblasts, colonic fibroblasts and CAFs will be evaluated using a triple quadrupole mass spectrometer, equipped with capillary flow electrospray ionization connected to a capillary pump. Standard peptides will be synthesized to contain traces of isotopes, and mixed with the samples, so that the baseline can be established for quantitation of the peptide concentration. The spectrometry data will be analyzed using Skyline, or other suitable software, to identify the molecules significantly upregulated or downregulated in CAFs.

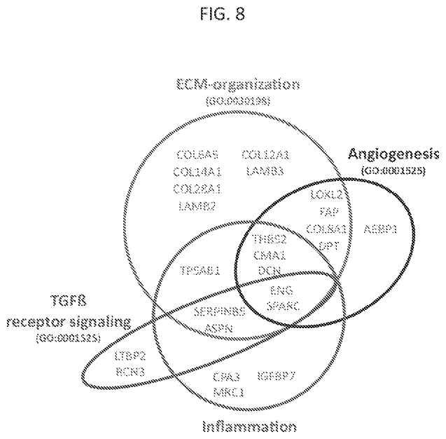

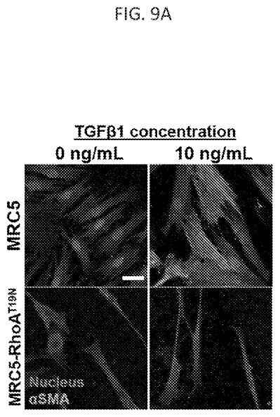

The molecules involved in signaling pathways leading to expression of known CAF markers/phenotypes are the potential candidates to be gnomically edited in STAR fibroblasts. The potential candidates identified by the mass spectrometry will be further verified by Western blot with appropriate antibodies. It is expected that molecules involved in TGF-β and STAT3 signaling are upregulated (FIG. 8). It is also expected that dermal and colonic fibroblasts exhibit similar protein expression. If multiple molecules involved in the same pathways are identified to be upregulated or downregulated in CAFs, the most downstream molecule in the pathway will be first considered to as the target of genomic editing. As a result, unanticipated inhibition of other pathways as the consequence of manipulating upstream molecules in a pathway can be avoided.

### Example 7

This example describes the generation of STAR fibroblasts by genomic editing.

To produce genetically engineered STAR fibroblasts, two sets of genes will be edited in the genome of the dermal fibroblasts. The first gene set includes ECM crosslinking enzymes, such as HAPLN1 and tissue transglutaminase (TG2), as well as genes observed to be downregulated in CAFs identified using the methods described in Example 6. HAPLN1 is a hyaluronic and proteoglycan link protein which crosslinks ECM fibrils. TG2 is a ECM crosslinking enzyme which ubiquitously expressed and also is secreted. The second gene set includes genes observed to be upregulated in CAFs, such as TGFBR1 and STAT3, or additional genes discovered using the methods described in Example 6. cDNAs of first set of genes will be introduced into the dermal fibroblasts to rescue the downregulation effects in CAFs. Appropriate promoters, such as EF1A, SV40 or PGK, will be used to drive strong, intermediate or weak expression, respectively, of the genes in the first set. The optimal choice of the promoter for each gene will be determined based on the efficacy of tumor suppression shown by genes identified in Example 6. The second sets of gene will be silenced in dermal fibroblasts using CRISPR/Cas 9. Electroporation (Lonza) will be used to deliver the cDNA constructs to reinforce expression of the first set of genes, and to introduce anti-sense RNA to knock out the second set of the genes, to the dermal fibroblasts. Moreover, to distinguish from the endogenous expression, the HAPLN1 gene will be fused with the DNA sequence encoding for FLAG, and the TG2 with His-Tag (Table 1).

TABLE 1
Target genes to be modified in STAR fibroblasts

| Target Gene | Function | Modulation |
|---|---|---|
| HAPLN1-Flag | ECM crosslinking | ↑ |
| TG2-His | ECM crosslinking | ↑ |
| Other Downregulated Genes in | — | ↑ |
| CAFs | | |
| TGFBR1 | TGFβ signaling | ↓ |
| STAT3 | STAT3 signaling | ↓ |
| Other Upregulated Genes in | — | ↓ |
| CAFs | | |

The dermal fibroblasts will be selected using antibiotics, for the vectors carrying the cDNAs also contain resistant genes against antibiotics. The selected cells will be used as described in Example 8.

### Example 8

This example describes the therapeutic efficacy of STAR fibroblasts.

Mice with CRC induced by AOM/DSS will be used to test the efficacy of STAR fibroblasts. STAR fibroblasts containing various promoters/cDNAs will be administered to the colon by rectal suppository when the mice are treated by carcinogens for 6 weeks. The DAI scores of CRC mice treated by STAR fibroblasts will be compared with the control mice without treatment and the mice treated with non-genetically engineered fibroblasts. The mice treated by STAR fibroblasts for 2, 3, 4, 6, and 8 weeks will be evaluated for the DAI score, survival rate, colon length, and tumor number. Furthermore, the colon will be dissected for histological examination. First, antibodies targeting FLAG or His-Tag will be used to assess the expression level of HAPLN1 or TG2, respectively. SCs, in the tissue sections, will be examined for CAF markers by immunofluorescence. The H&E staining will be also performed in the tissue sections to examine ECM architecture. The orientation of ECM fibrils will be quantified as described in above. It is expected that the ECM alignment and the CAF marker expression will be reduced compared to the controls. The promoters/cDNAs combination in STAR fibroblasts with the most effective tumor suppression effect will be used for future studies.

It is still yet to be assessed how long STAR fibroblasts remain viable in TME. It is possible that the continuous secretion of crosslinking enzymes significantly stiffens ECM, activating mechanosignaling pathways that are known to promote tumor progression. In that case, inducible promoter(s) will be used to control the gene expression of ECM crosslinking enzymes. Tet-on promotor is one such promoter to be considered, which upon the addition of doxycycline will drive gene expression on-demand. The optimal dosage, and the frequency of the doxycycline can be determined by measuring the stiffness of ECM as described above.

### Example 9

This example demonstrates that prototype STAR fibroblasts can suppress tumor growth, cancer cell invasion, and the development of drug resistant in cancer cells in a 3D cell culture model.

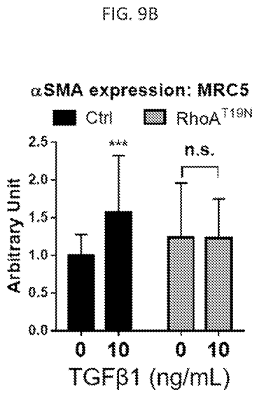

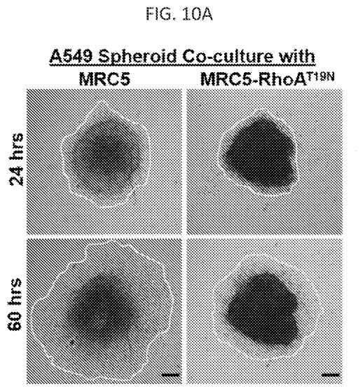

Prototype STAR fibroblasts were generated by introducing the dominant negative form of RhoA (RhoA^{T19N}) into to human lung fibroblasts (MRCS) to reduce RhoA signaling, a key mediator for CAF phenotypes. As shown in FIGS. 9A and 9B, the expression of CAF marker αSMA did not increase in the prototype STAR fibroblasts after TGFβ1 (10 ng/mL) treatment when control fibroblasts showed 50% increase (p<0.001) compared with the non-treated group.

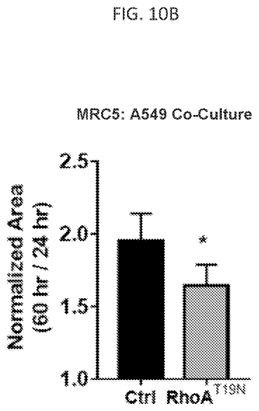

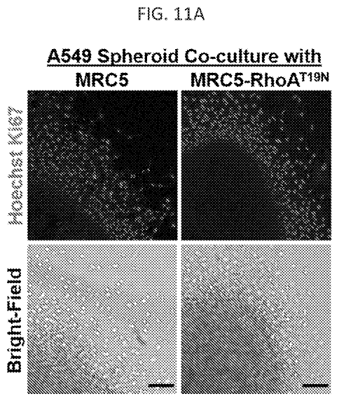

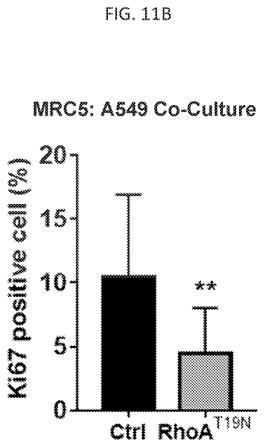

A tumor spheroid consisting of human lung cancer cells (A549) were embedded in a 3D collagen matrix and co-cultured with prototype STAR fibroblasts, or with control MRCS fibroblasts, for 60 hours. Cancer cell invasion was quantified as the ratio of the spreading area at 24 hours and at 60 hours of cocultures. The percentage of cancer cells expressing Ki67, a cell proliferation marker, also was quantified. The prototype STAR fibroblasts suppressed cancer invasion by approximately 40% (p<0.05), as shown in FIGS. 10A and 10B. Proliferation of lung cancer cells co-cultured with prototype STAR fibroblasts decreased to 50% (p<0.01) of that observed in the control groups, as shown in FIGS. 11A and 11B.

The tumor spheroid consisting of human lung cancer cells (A549) were embedded in 3D collagen matrix and co-cultured with prototype STAR fibroblasts, or with control MRCS fibroblasts, for 60 hours. Nucleus (blue) is stained with Hoechst dye. Scale bar: 100 μm.

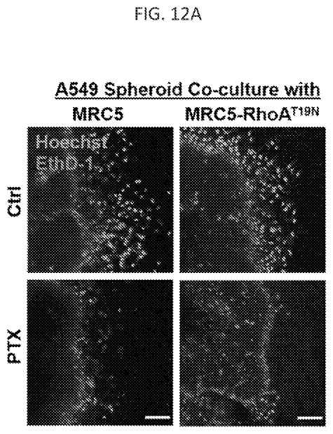

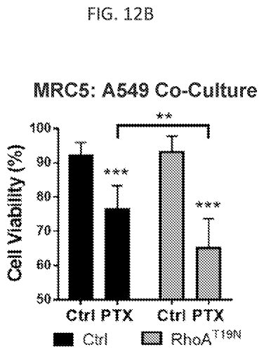

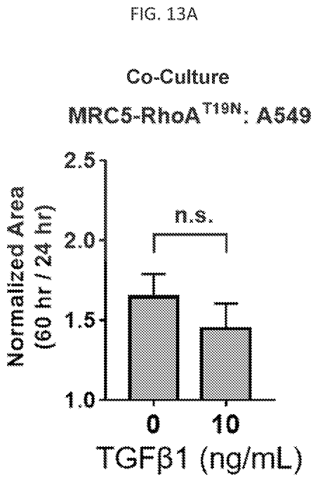

In a separate experiment, after 24 hours of co-culturing A549 tumor spheroids with prototype STAR fibroblasts or MRCS control fibroblasts in a 3D collagen matrix, 100 nM of paclitaxel (PTX), a commonly used chemotherapeutic drug for anti-cancer treatment, was added to the co-culture system. The cancer cell viability was examined after another 24 hours by staining the samples with EthD-1 dye (red) and Hoechst (green). The presence of live cells after PTX treatment indicated the development of PTX-resistance. On average, 15% less PTX-resistant cells were observed in the cocultures containing prototype STAR fibroblasts (p<0.01), as shown in FIGS. 12A and 12B.

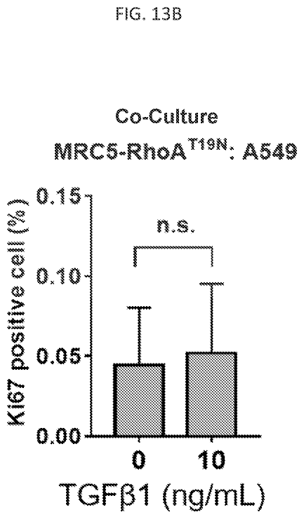

The prototype STAR fibroblasts were then treated with 0 or 10 ng/mL TG931 prior to co-culturing with A549 cells lung cancer cells. As shown in FIGS. 13A and 13B, TG931-treated prototype STAR fibroblasts did not result in increased cancer invasion or proliferation, suggesting the prototype STAR fibroblasts will not be induced to behave as CAFs and facilitate tumor progression.

### REFERENCES

1. Jung, W.-H., Elawad, K., Kang, S. H. & Chen, Y. Cell—Cell Adhesion and Myosin Activity Regulate Cortical Actin Assembly in Mammary Gland Epithelium on Concaved Surface. Cells 8, 813 (2019).
2. Park, S., Lui, C., Jung, W., Maity, D., Ong, C. S., Bush, J., Maruthamuthu, V., Hibino, N. & Chen, Y. Mechanical Characterization of hiPSC-Derived Cardiac Tissues for Quality Control. Adv. Biosyst. 2, 1800251 (2018).
3. Cherry, S., Jin, E. J., Özel, M. N., Lu, Z., Agi, E., Wang, D., Jung, W. H., Epstein, D., Meinertzhagen, I. A., Chan, C. C. & Robin Hiesinger, P. Charcot-Marie-Tooth 2B mutations in rab7 cause dosage-dependent neurodegeneration due to partial loss of function. Elife 2013, (2013).
4. Jung, W., Liu, C., Yu, Y., Chang, Y., Lien, W., Chao, H., Huang, S., Kuo, C., Ho, H. & Chan, C. Lipophagy prevents activity-dependent neurodegeneration due to dihydroceramide accumulation in vivo. EMBO Rep. e201643480 (2017). doi:10.15252/embr.201643480
5. Yang, C. N., Wu, M. F., Liu, C. C., Jung, W. H., Chang, Y. C., Lee, W. P., Shiao, Y. J., Wu, C. L., Liou, H. H., Lin, S. K. & Chan, C. C. Differential protective effects of connective tissue growth factor against AP neurotoxicity on neurons and glia. Hum. Mol. Genet. 26, 3909-3921 (2017).
6. Bray, F., Ferlay, J., Soerjomataram, I., Siegel, R. L., Torre, L. A. & Jemal, A. Global cancer statistics 2018: GLOBOCAN estimates of incidence and mortality worldwide for 36 cancers in 185 countries. CA. Cancer J. Clin. 68, 394-424 (2018).
7. Moiel, D. Early Detection of Colon Cancer—The Kaiser Permanente Northwest 30-Year History: How Do We Measure Success? Is It the Test, the Number of Tests, the Stage, or the Percentage of Screen-Detected Patients. Perm. J. 15, (2011).
8. Survival rates for colorectal cancer. at <https://www.cancer.org/cancer/colon-rectal-cancer/detection-diagnosis-staging/survival-rates.html>
9. Quail, D. F. & Joyce, J. A. Microenvironmental regulation of tumor progression and metastasis. Nat. Med. 19, 1423-1437 (2013).
10. Crotti, S., Piccoli, M., Rizzolio, F., Giordano, A., Nitti, D. & Agostini, M. Extracellular Matrix and Colorectal Cancer: How Surrounding Microenvironment Affects Cancer Cell Behavior? J. Cell. Physiol. 232, 967-975 (2017).
11. Provenzano, P. P., Eliceiri, K. W., Campbell, J. M., Inman, D. R., White, J. G. & Keely, P. J. Collagen reorganization at the tumor-stromal interface facilitates local invasion. BMC Med. 4, 38 (2006).
12. Cho, A., Howell, V. M. & Colvin, E. K. The Extracellular Matrix in Epithelial Ovarian Cancer—A Piece of a Puzzle. Front. Oncol. 5, (2015).
13. Siegel, R. L., Miller, K. D. & Jemal, A. Cancer statistics, 2019. CA. Cancer J. Clin. 69, 7-34 (2019).
14. Abulafi, A. M. & Williams, N. S. Local recurrence of colorectal cancer: The problem, mechanisms, management and adjuvant therapy. Br. J. Surg. 81, 7-19 (1994).
15. Saigusa, S., Toiyama, Y., Tanaka, K., Yokoe, T., Okugawa, Y., Fujikawa, H., Matsusita, K., Kawamura, M., Inoue, Y., Miki, C. & Kusunoki, M. Cancer-associated fibroblasts correlate with poor prognosis in rectal cancer after chemoradiotherapy. Int. J. Oncol. 38, 655-63 (2011).
16. Erdogan, B., Ao, M., White, L. M., Means, A. L., Brewer, B. M., Yang, L., Washington, M. K., Shi, C., Franco, 0. E., Weaver, A. M., Hayward, S. W., Li, D. & Webb, D. J. Cancer-associated fibroblasts promote directional cancer cell migration by aligning fibronectin. J. Cell Biol. 216, 3799-3816 (2017).
17. Attieh, Y., Clark, A. G., Grass, C., Richon, S., Pocard, M., Mariani, P., Elkhatib, N., Betz, T., Gurchenkov, B. & Vignjevic, D. M. Cancer-associated fibroblasts lead tumor invasion through integrin-β3-dependent fibronectin assembly. J. Cell Biol. 216, 3509-3520 (2017).
18. Karnoub, A. E., Dash, A. B., Vo, A. P., Sullivan, A., Brooks, M. W., Bell, G. W., Richardson, A. L., Polyak, K., Tubo, R. & Weinberg, R. A. Mesenchymal stem cells within tumour stroma promote breast cancer metastasis. Nature 449, 557-63 (2007).
19. Ying, L., Zhu, Z., Xu, Z., He, T., Li, E., Guo, Z., Liu, F., Jiang, C. & Wang, Q. Cancer associated fibroblast-derived hepatocyte growth factor inhibits the paclitaxel-induced apoptosis of lung cancer A549 cells by up-regulating the PI3K/Akt and GRP78 signaling on a microfluidic platform. PLoS One 10, e0129593 (2015).
20. Rösel, D., Brábek, J., Tolde, O., Mierke, C. T., Zitterbart, D. P., Raupach, C., Bicanová, K., Kollmannsberger, P., Paňková, D., Veselý, P., Folk, P. & Fabry, B. Up-Regulation of Rho/ROCK Signaling in Sarcoma Cells Drives Invasion and Increased Generation of Protrusive Forces. Mol. Cancer Res. 6, (2008).
21. Kraning-Rush, C. M., Califano, J. P. & Reinhart-King, C. A. Cellular Traction Stresses Increase with Increasing Metastatic Potential. PLoS One 7, e32572 (2012).
22. Borges, F. T., Melo, S. A., Özdemir, B. C., Kato, N., Revuelta, I., Miller, C. a, Gattone, V. H., LeBleu, V. S. & Kalluri, R. TGF-β1-Containing Exosomes from Injured Epithelial Cells Activate Fibroblasts to Initiate Tissue Regenerative Responses and Fibrosis. J. Am. Soc. Nephrol. 24, 385-392 (2013).
23. Chiba, M., Kimura, M. & Asari, S. Exosomes secreted from human colorectal cancer cell lines contain mRNAs, microRNAs and natural antisense RNAs, that can transfer into the human hepatoma HepG2 and lung cancer A549 cell lines. Oncol. Rep. 28, 1551-1558 (2012).
24. Valadi, H., Ekström, K., Bossios, A., Sjöstrand, M., Lee, J. J. & Lötvall, J. O. Exosome-mediated transfer of mRNAs and microRNAs is a novel mechanism of genetic exchange between cells. Nat. Cell Biol. 9, 654-9 (2007).
25. Schindelin, J., Arganda-Carreras, I., Frise, E., Kaynig, V., Longair, M., Pietzsch, T., Preibisch, S., Rueden, C., Saalfeld, S., Schmid, B., Tinevez, J.-Y., White, D. J., Hartenstein, V., Eliceiri, K., Tomancak, P. & Cardona, A. Fiji: an open-source platform for biological-image analysis. Nat. Methods 9, 676-682 (2012).
26. Fonck, E., Feigl, G. G., Fasel, J., Sage, D., Unser, M., Rüfenacht, D. A. & Stergiopulos, N. Effect of aging on elastin functionality in human cerebral arteries. Stroke 40, 2552-2556 (2009).
27. Sappino, A.-P., Skalli, O., Jackson, B., Schürch, W. & Gabbiani, G. Smooth-muscle differentiation in stromal cells of malignant and non-malignant breast tissues. Int. J. Cancer 41, 707-712 (1988).
28. Mishra, P. J., Mishra, P. J., Humeniuk, R., Medina, D. J., Alexe, G., Mesirov, J. P., Ganesan, S., Glod, J. W. & Banerjee, D. Carcinoma-Associated Fibroblast-Like Differentiation of Human Mesenchymal Stem Cells. Cancer Res. 68, 4331-4339 (2008).
29. Gascard, P. & Tlsty, T. D. Carcinoma-associated fibroblasts: orchestrating the composition of malignancy. Genes Dev. 30, 1002-1019 (2016).
30. Stadler, S., Nguyen, C. H., Schachner, H., Milovanovic, D., Holzner, S., Brenner, S., Eichsteininger, J., Stadler, M., Senfter, D., Krenn, L., Schmidt, W. M., Huttary, N., Krieger, S., Koperek, O., Bago-Horvath, Z., Brendel, K. A., Marian, B., de Wever, O., Mader, R. M., Giessrigl, B., Jäger, W., Dolznig, H. & Krupitza, G. Colon cancer cell-derived 12(S)-HETE induces the retraction of cancer-associated fibroblast via MLC2, RHO/ROCK and Ca2+ signalling. Cell. Mol. Life Sci. 74, 1907-1921 (2017).
31. Calvo, F., Ege, N., Grande-Garcia, A., Hooper, S., Jenkins, R. P., Chaudhry, S. I., Harrington, K., Williamson, P., Moeendarbary, E., Charras, G. & Sahai, E. Mechanotransduction and YAP-dependent matrix remodelling is required for the generation and maintenance of cancer-associated fibroblasts. Nat. Cell Biol. 15, 637-646 (2013).
32. Li, Q., Zhang, D., Wang, Y., Sun, P., Hou, X., Lamer, J., Xiong, W. & Mi, J. MiR-21/Smad 7 signaling determines TGF-β1-induced CAF formation. Sci. Rep. 3, 2038 (2013).
33. Ringuette Goulet, C., Bernard, G., Tremblay, S., Chabaud, S., Bolduc, S. & Pouliot, F. Exosomes Induce Fibroblast Differentiation into Cancer-Associated Fibroblasts through TGFβ Signaling. Mol. Cancer Res. 16, 1196-1204 (2018).
34. Fang, T., Lv, H., Lv, G., Li, T., Wang, C., Han, Q., Yu, L., Su, B., Guo, L., Huang, S., Cao, D., Tang, L., Tang, S., Wu, M., Yang, W. & Wang, H. Tumor-derived exosomal miR-1247-3p induces cancer-associated fibroblast activation to foster lung metastasis of liver cancer. Nat. Commun. 9, 191 (2018).
35. Stylianopoulos, T., Diop-Frimpong, B., Munn, L. L. & Jain, R. K. Diffusion Anisotropy in Collagen Gels and Tumors: The Effect of Fiber Network Orientation. Biophys. J. 99, 3119-3128 (2010).
36. Vardaki, I., Ceder, S., Rutishauser, D., Baltatzis, G., Foukakis, T. & Panaretakis, T. Periostin is identified as a putative metastatic marker in breast cancer-derived exosomes. Oncotarget 7, 74966-74978 (2016).
37. Levental, K. R., Yu, H., Kass, L., Lakins, J. N., Egeblad, M., Erler, J. T., Fong, S. F. T., Csiszar, K., Giaccia, A., Weninger, W., Yamauchi, M., Gasser, D. L. & Weaver, V. M. Matrix Crosslinking Forces Tumor Progression by Enhancing Integrin Signaling. Cell 139, 891-906 (2009).
38. Zhang, X., Chen, X., Yang, T., Zhang, N., Dong, L., Ma, S., Liu, X., Zhou, M. & Li, B. The effects of different crossing-linking conditions of genipin on type I collagen scaffolds: an in vitro evaluation. Cell Tissue Bank. 15, 531-541 (2014).
39. Orban, J. M., Wilson, L. B., Kofroth, J. A., El-Kurdi, M. S., Maul, T. M. & Vorp, D. A. Crosslinking of collagen gels by transglutaminase. J. Biomed. Mater. Res. 68A, 756-762 (2004).
40. Kaur, A., Ecker, B. L., Douglass, S. M., Kugel, C. H., Webster, M. R., Almeida, F. V., Somasundaram, R., Hayden, J., Ban, E., Ahmadzadeh, H., Franco-Barraza, J., Shah, N., Mellis, I. A., Keeney, F., Kossenkov, A., Tang, H.-Y., Yin, X., Liu, Q., Xu, X., Fane, M., Brafford, P., Herlyn, M., Speicher, D. W., Wargo, J. A., Tetzlaff, M. T., Haydu, L. E., Raj, A., Shenoy, V., Cukierman, E. & Weeraratna, A. T. Remodeling of the Collagen Matrix in Aging Skin Promotes Melanoma Metastasis and Affects Immune Cell Motility. Cancer Discov. 9, 64-81 (2019).
41. Shanmugam, M. K., Shen, H., Tang, F. R., Arfuso, F., Rajesh, M., Wang, L., Kumar, A. P., Bian, J., Goh, B. C., Bishayee, A. & Sethi, G. Potential role of genipin in cancer therapy. Pharmacol. Res. 133, 195-200 (2018).
42. Lorand, L. & Graham, R. M. Transglutaminases: Crosslinking enzymes with pleiotropic functions. Nat. Rev. Mol. Cell Biol. 4, 140-156 (2003).
43. Yu, H., Mouw, J. K. & Weaver, V. M. Forcing form and function: Biomechanical regulation of tumor evolution. Trends Cell Biol. 21, 47-56 (2011).
44. Zaidel-Bar, R., Zhenhuan, G. & Luxenburg, C. The contractome—A systems view of actomyosin contractility in non-muscle cells. J. Cell Sci. 128, 2209-2217 (2015).
45. Tanaka, T., Kohno, H., Suzuki, R., Yamada, Y., Sugie, S. & Mori, H. A novel inflammation-related mouse colon carcinogenesis model induced by azoxymethane and dextran sodium sulfate. Cancer Sci. 94, 965-973 (2003).
46. De Robertis, M., Massi, E., Poeta, M. L., Carotti, S., Morini, S., Cecchetelli, L., Signori, E. & Fazio, V. M. The AOM/DSS murine model for the study of colon carcinogenesis: From pathways to diagnosis and therapy studies. J. Carcinog. 10, 9 (2011).
47. Ngan, C. Y., Yamamoto, H., Seshimo, I., Tsujino, T., Man-i, M., Ikeda, J.-I., Konishi, K., Takemasa, I., Ikeda, M., Sekimoto, M., Matsuura, N. & Monden, M. Quantitative evaluation of vimentin expression in tumour stroma of colorectal cancer. Br. J. Cancer 96, 986-92 (2007).
48. Beningo, K. A., Hamao, K., Dembo, M., Wang, Y.-L. & Hosoya, H. Traction forces of fibroblasts are regulated by the Rho-dependent kinase but not by the myosin light chain kinase. Arch. Biochem. Biophys. 456, 224-31 (2006).
49. Abraham, S. & Srinath, M. Development of modified pulsincap drug delivery system of metronidazole for drug targeting. Indian J. Pharm. Sci. 69, 24 (2007).
50. Krögel, I. & Bodmeier, R. Pulsatile drug release from an insoluble capsule body controlled by an erodible plug. Pharm. Res. 15, 474-81 (1998).
51. Chavez, J. C., Bachmeier, C. & Kharfan-Dabaja, M. A. CAR T-cell therapy for B-cell lymphomas: clinical trial results of available products. Ther. Adv. Hematol. 10, 204062071984158 (2019).
52. Wei, X., Yang, X., Han, Z. P., Qu, F. F., Shao, L. & Shi, Y. F. Mesenchymal stem cells: A new trend for cell therapy. Acta Pharmacol. Sin. 34, 747-754 (2013).
53. François, S., Usunier, B., Forgue-Lafitte, M.-E., L'Homme, B., Benderitter, M., Douay, L., Gorin, N.-C., Larsen, A. K. & Chapel, A. Mesenchymal Stem Cell Administration Attenuates Colon Cancer Progression by Modulating the Immune Component within the Colorectal Tumor Microenvironment. Stem Cells Transl. Med. 8, 285-300 (2019).
54. Ichim, T. E., O'Heeron, P. & Kesari, S. Fibroblasts as a practical alternative to mesenchymal stem cells. J. Transl. Med. 16, (2018).
55. Koliaraki, V., Roulis, M. & Kollias, G. Tp12 regulates intestinal myofibroblast HGF release to suppress colitis-associated tumorigenesis. J. Clin. Invest. 122, 4231-4242 (2012).
56. Zempel, G. & Hulser, D. Growth Inhibition of Oncogene-transformed Rat Fibroblasts by Cocultured Normal Cells: Relevance of Metabolic Cooperation Mediated by Gap Junctions1. Cancer Res. 51, 5348-5354 (1991).
57. Allard, D., Stoker, M. & Gherardi, E. A G2/M cell cycle block in transformed cells by contact with normal neighbors. Cell Cycle 2, 484-7 (2003).
58. Kirk, D., Szalay, M. F. & Kaighn, M. E. Modulation of growth of a human prostatic cancer cell line (PC-3) in agar culture by normal human lung fibroblasts. Cancer Res. 41, 1100-3 (1981).
59. Petrof, G., Martinez-Queipo, M., Mellerio, J. E., Kemp, P. & McGrath, J. A. Fibroblast cell therapy enhances initial healing in recessive dystrophic epidermolysis bullosa wounds: Results of a randomized, vehicle-controlled trial. Br. J. Dermatol. 169, 1025-1033 (2013).
60. Venugopal, S. S., Yan, W., Frew, J. W., Cohn, H. I., Rhodes, L. M., Tran, K., Melbourne, W., Nelson, J. A., Sturm, M., Fogarty, J., Marinkovich, M. P., Igawa, S., Ishida-Yamamoto, A. & Murrell, D. F. A phase II randomized vehicle-controlled trial of intradermal allogeneic fibroblasts for recessive dystrophic epidermolysis bullosa. J. Am. Acad. Dermatol. 69, (2013).
61. Hart, C. E., Loewen-Rodriguez, A. & Lessem, J. Dermagraft: Use in the Treatment of Chronic Wounds. Adv. Wound Care 1, 138-141 (2012).
62. Munavalli, G. S., Smith, S., Maslowski, J. M. & Weiss, R. A. Successful treatment of depressed, distensible acne scars using autologous fibroblasts: A multi-site, prospective, double blind, placebo-controlled clinical trial. Dermatologic Surg. 39, 1226-1236 (2013).
63. Hawinkels, L. J. A. C., Paauwe, M., Verspaget, H. W., Wiercinska, E., Van Der Zon, J. M., Van Der Ploeg, K., Koelink, P. J., Lindeman, J. H. N., Mesker, W., Ten Dijke, P. & Sier, C. F. M. Interaction with colon cancer cells hyperactivates TGF-β signaling in cancer-associated fibroblasts. Oncogene 33, 97-107 (2014).
64. Peddareddigari, V. G., Wang, D. & Dubois, R. N. The tumor microenvironment in colorectal carcinogenesis. in Cancer Microenviron. 3, 149-166 (2010).
65. Albrengues, J., Bourget, I., Pons, C., Butet, V., Hofman, P., Tartare-Deckert, S., Feral, C. C., Meneguzzi, G. & Gaggioli, C. LIF mediates proinvasive activation of stromal fibroblasts in cancer. Cell Rep. 7, 1664-1678 (2014).
66. Drev, D., Bileck, A., Erdem, Z. N., Mohr, T., Timelthaler, G., Beer, A., Gerner, C. & Marian, B. Proteomic profiling identifies markers for inflammation-related tumor-fibroblast interaction. Clin. Proteomics 14, (2017).
67. Mevizou, R., Sirvent, A. & Roche, S. Control of tyrosine kinase signalling by small adaptors in colorectal cancer. Cancers (Basel). 11, (2019).
68. Khan, M. & Gasser, S. Generating primary fibroblast cultures from mouse ear and tail tissues. J. Vis. Exp. 2016, (2016).
69. Cañas Montalvo, B., López-Ferrer, D., Ramos-Fernández, A., Camafeita, E. & Calvo, E. Mass spectrometry technologies for proteomics. Briefings Funct. Genomics Proteomics 4, 295-320 (2006).
70. MacLean, B., Tomazela, D. M., Shulman, N., Chambers, M., Finney, G. L., Frewen, B., Kern, R., Tabb, D. L., Liebler, D. C. & MacCoss, M. J. Skyline: an open source document editor for creating and analyzing targeted proteomics experiments. Bioinformatics 26, 966-968 (2010).
71. Martens, M. F. W. C., Huyben, C. M. L. C. & Hendriks, T. Collagen synthesis in fibroblasts from human colon: Regulatory aspects and differences with skin fibroblasts. Gut 33, 1664-1670 (1992).
72. Bains, W. Transglutaminse 2 and EGGL, the protein cross-link formed by transglutaminse 2, as therapeutic targets for disabilities of old age. Rejuvenation Res. 16, 495-517 (2013).
73. Qin, J. Y., Zhang, L., Clift, K. L., Hulur, I., Xiang, A. P., Ren, B.-Z. & Lahn, B. T. Systematic Comparison of Constitutive Promoters and the Doxycycline-Inducible Promoter. PLoS One 5, e10611 (2010).
74. Broders-Bondon, F., Ho-Bouldoires, T. H. N., Fernandez-Sanchez, M. E. & Farge, E. Mechanotransduction in tumor progression: The dark side of the force. J. Cell Biol. 217, 1571-1587 (2018).
75. Navab, R. et al. Prognostic gene-expression signature of carcinoma-associated fibroblasts in non-small cell lung cancer. Proc. Natl. Acad. Sci. 108, 7160-7165 (2011).
76. Takahashi, Y. et al. Fibrous stroma is associated with poorer prognosis in lung squamous cell carcinoma patients. J. Thorac. Oncol. 6, 1460-1467 (2011).
77. Paulsson, J. & Micke, P. Prognostic relevance of cancer-associated fibroblasts in human cancer. Seminars in Cancer Biology 25, 61-68 (2014).
78. Erdogan, B. et al. Cancer-associated fibroblasts promote directional cancer cell migration by aligning fibronectin. J. Cell Biol. 216, 3799-3816 (2017).
79. Attieh, Y. et al. Cancer-associated fibroblasts lead tumor invasion through integrin-f33-dependent fibronectin assembly. J. Cell Biol. 216, 3509-3520 (2017).
80. Karnoub, A. E. et al. Mesenchymal stem cells within tumour stroma promote breast cancer metastasis. Nature 449, 557-63 (2007).
81. Ying, L. et al. Cancer associated fibroblast-derived hepatocyte growth factor inhibits the paclitaxel-induced apoptosis of lung cancer A549 cells by up-regulating the PI3K/Akt and GRP78 signaling on a microfluidic platform. PLoS One 10, e0129593 (2015).
82. Sato, N., Maehara, N. & Goggins, M. Gene expression profiling of tumor-stromal interactions between pancreatic cancer cells and stromal fibroblasts. Cancer Res. 64, 6950-6 (2004).
83. Olsen, C. J., Moreira, J., Lukanidin, E. M. & Ambartsumian, N. S. Human mammary fibroblasts stimulate invasion of breast cancer cells in a three-dimensional culture and increase stroma development in mouse xenografts. BMC Cancer 10, 444 (2010).
84. Borges, F. T. et al. TGF-β1-Containing Exosomes from Injured Epithelial Cells Activate Fibroblasts to Initiate Tissue Regenerative Responses and Fibrosis. J. Am. Soc. Nephrol. 24, 385-392 (2013).
85. Chiba, M., Kimura, M. & Asari, S. Exosomes secreted from human colorectal cancer cell lines contain mRNAs, microRNAs and natural antisense RNAs, that can transfer into the human hepatoma HepG2 and lung cancer A549 cell lines. Oncol. Rep. 28, 1551-1558 (2012).
86. Valadi, H. et al. Exosome-mediated transfer of mRNAs and microRNAs is a novel mechanism of genetic exchange between cells. Nat. Cell Biol. 9, 654-9 (2007).
87. Théry, C. Exosomes: secreted vesicles and intercellular communications. F1000 Biol.

Rep. 3, 15 (2011).
88. Rizki, A. et al. A Human Breast Cell Model of Preinvasive to Invasive Transition. Cancer Res. 68, 1378-1387 (2008).
89. Ma, X.-J., Dahiya, S., Richardson, E., Erlander, M. & Sgroi, D. C. Gene expression profiling of the tumor microenvironment during breast cancer progression. Breast Cancer Res. 11, R7 (2009).
90. Tester, A. M., Ruangpanit, N., Anderson, R. L. & Thompson, E. W. MMP-9 secretion and MMP-2 activation distinguish invasive and metastatic sublines of a mouse mammary carcinoma system showing epithelialmesenchymal transition traits. Clin. Exp. Metastasis 18, 553-60 (2000).
91. Verghese, E. T. et al. MiR-26b is down-regulated in carcinoma-associated fibroblasts from ER-positive breast cancers leading to enhanced cell migration and invasion. J. Pathol. 231, 388-399 (2013).
92. Sappino, A.-P., Skalli, O., Jackson, B., Schürch, W. & Gabbiani, G. Smooth-muscle differentiation in stromal cells of malignant and non-malignant breast tissues. Int. J. Cancer 41, 707-712 (1988).
93. Mishra, P. J. et al. Carcinoma-Associated Fibroblast-Like Differentiation of Human Mesenchymal Stem Cells. Cancer Res. 68, 4331-4339 (2008).
94. Tao, K., Fang, M., Alroy, J. & Sahagian, G. G. Imagable 4T1 model for the study of late stage breast cancer. BMC Cancer 8,228 (2008).
95. Fonck, E. et al. Effect of aging on elastin functionality in human cerebral arteries. Stroke 40, 2552-2556 (2009).
96. Li, Q. et al. MiR-21/Smad 7 signaling determines TGF-β1-induced CAF formation. Sci. Rep. 3,2038 (2013).
97. Ringuette Goulet, C. et al. Exosomes Induce Fibroblast Differentiation into Cancer-Associated Fibroblasts through TGFβ Signaling. Mol. Cancer Res. 16, 1196-1204 (2018).
98. Fang, T. et al. Tumor-derived exosomal miR-1247-3p induces cancer-associated fibroblast activation to foster lung metastasis of liver cancer. Nat. Commun. 9,191 (2018).
99. Vardaki, I. et al. Periostin is identified as a putative metastatic marker in breast cancer-derived exosomes. Oncotarget 7, 74966-74978 (2016).
100. Stylianopoulos, T., Diop-Frimpong, B., Munn, L. L. & Jain, R. K. Diffusion Anisotropy in Collagen Gels and Tumors: The Effect of Fiber Network Orientation. Biophys. J. 99, 3119-3128 (2010).
101. Bigi, A., Cojazzi, G., Panzavolta, S., Roveri, N. & Rubini, K. Stabilization of gelatin films by crosslinking with genipin. Biomaterials 23, 4827-4832 (2002).
102. Sundararaghavan, H. G. et al. Genipin-induced changes in collagen gels: Correlation of mechanical properties to fluorescence. J. Biomed. Mater. Res. Part A 87A, 308-320 (2008).
103. Zhang, X. et al. The effects of different crossing-linking conditions of genipin on type I collagen scaffolds: an in vitro evaluation. Cell Tissue Bank. 15, 531-541 (2014).
104. Wipff, P. J., Rifkin, D. B., Meister, J. J. & Hinz, B. Myofibroblast contraction activates latent TGF-β1 from the extracellular matrix. J. Cell Biol. 179, 1311-1323 (2007).
105. Iwanicki, M. P. et al. Ovarian cancer spheroids use myosin-generated force to clear the mesothelium. Cancer Discov. 1, 144-57 (2011).
106. Subauste, M. C. et al. Rho Family Proteins Modulate Rapid Apoptosis Induced by Cytotoxic T Lymphocytes and Fas. J. Biol. Chem. 275, 9725-9733 (2000).
107. Beningo, K. A., Hamao, K., Dembo, M., Wang, Y.-L. & Hosoya, H. Traction forces of fibroblasts are regulated by the Rho-dependent kinase but not by the myosin light chain kinase. Arch. Biochem. Biophys. 456, 224-31 (2006).
108. Shiu, Y.-T. et al. Rho Mediates the Shear-Enhancement of Endothelial Cell Migration and Traction Force Generation. Biophys. J. 86, 2558-2565 (2004).
109. Riching, K. M. et al. 3D Collagen Alignment Limits Protrusions to Enhance Breast Cancer Cell Persistence. Biophys. J. 107, 2546-2558 (2014).
110. Calvo, F. et al. Mechanotransduction and YAP-dependent matrix remodelling is required for the generation and maintenance of cancer-associated fibroblasts. Nat. Cell Biol. 15, 637-46 (2013).
111. Sulzmaier, F. J., Jean, C. & Schlaepfer, D. D. FAK in cancer: mechanistic findings and clinical applications. Nat. Rev. Cancer 14, 598-610 (2014).
112. Kalluri, R. & Zeisberg, M. Fibroblasts in cancer. Nat. Rev. Cancer 6, 392-401 (2006).
113. Gascard, P. & Tlsty, T. D. Carcinoma-associated fibroblasts: orchestrating the composition of malignancy. Genes Dev. 30, 1002-1019 (2016).
114. Kelm, J. M., Timmins, N. E., Brown, C. J., Fussenegger, M. & Nielsen, L. K. Method for generation of homogeneous multicellular tumor spheroids applicable to a wide variety of cell types. Biotechnol. Bioeng. 83, 173-180 (2003).
115. Taufalele, P. V., VanderBurgh, J. A., Muñoz, A., Zanotelli, M. R. & Reinhart-King, C. A. Fiber alignment drives changes in architectural and mechanical features in collagen matrices. PLoS One 14, e0216537 (2019).
116. Djerassi, C., Gray, J. D. & Kincl, F. A. Naturally Occurring Oxygen Heterocyclics. IX.1 Isolation and Characterization of Genipin2. J. Org. Chem. 25, 2174-2177 (1960).
117. Style, R. W. et al. Traction force microscopy in physics and biology. Soft Matter 10, 4047-4055 (The Royal Society of Chemistry, 2014).
118. Gutierrez, E. et al. High refractive index silicone gels for simultaneous total internal reflection fluorescence and traction force microscopy of adherent cells. PLoS One 6, e23807 (2011).
119. Tseng, Q. et al. Spatial organization of the extracellular matrix regulates cell-cell junction positioning. Proc. Natl. Acad. Sci. 109, 1506-1511 (2012).
120. Piccinini, F., Kiss, A. & Horvath, P. CellTracker (not only) for dummies. Bioinformatics 32, 955-957 (2016).
121. Schindelin, J. et al. Fiji: an open-source platform for biological-image analysis. Nat. Methods 9, 676-682 (2012).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising fibroblasts that have been genetically engineered to (a) express one or more genes that are downregulated in carcinoma-associated fibroblasts (CAFs) and/or (b) silence one or more genes that are upregulated in cancer CAFs.

2. The composition of claim 1, wherein the one or more genes that are downregulated in carcinoma-associated fibroblasts are one or more extracellular matrix crosslinking genes.

3. The composition of claim 2, wherein the one or more ECM crosslinking genes are HAPLN1, genipin, and/or tissue transglutaminase (TG2).

4. The composition of any one of claims 1-3, wherein the one or more genes that are upregulated in carcinoma-associated fibroblasts are TGFBR1 and/or STAT3.

5. A method of inhibiting extracellular matrix (ECM) remodeling in the microenvironment of a cancerous tumor, which comprises administering the composition of any one of claims 1-4 to the microenvironment of a cancerous tumor, whereupon one more genes downregulated in CAFs are expressed and/or one or more genes upregulated in cancer CAFs are silenced in the microenvironment of the cancerous tumor, thereby inhibiting ECM remodeling in the microenvironment of the cancerous tumor.

6. A method of inhibiting progression of a cancerous tumor, which comprises administering the composition of any one of claims 1-4 to the microenvironment of a cancerous tumor, whereupon one more genes downregulated in CAFs are expressed and/or one or more genes upregulated in cancer CAFs are silenced in the microenvironment of the cancerous tumor, thereby inhibiting progression of the cancerous tumor.

7. The method of claim 5 or claim 6, wherein the method is performed in vivo.

8. The method of claim 7, wherein the method is performed in a human.

9. The method of claim 5 or claim 6, wherein the method is performed ex vivo.

10. A method of inhibiting progression of a cancerous tumor, which method comprises:
(a) genetically engineering a population of fibroblasts obtained from a subject to (a) express one or more genes that are downregulated in carcinoma-associated fibroblasts (CAFs) and/or (b) silence one or more genes that are upregulated in cancer CAFs; and
(b) administering the genetically engineered population of fibroblasts to the microenvironment of a cancerous tumor present in the subject, whereupon the one more genes downregulated in CAFs are expressed and/or the one or more genes upregulated in cancer CAFs are silenced in the microenvironment of the cancerous tumor, thereby inhibiting progression of the cancerous tumor in the subject.

11. The method of claim 10, wherein the population of fibroblasts are genetically engineered using gene editing.

12. The method of claim 11, wherein gene editing is performed using a CRISPR/Cas system.

13. The method of any one of claims 10-12, wherein the one or more genes that are downregulated in carcinoma-associated fibroblasts are one or more extracellular matrix crosslinking genes.

14. The method of claim 13, wherein the one or more ECM crosslinking genes are HAPLN1, genipin, and/or tissue transglutaminase (TG2).

15. The method of any one of claims 10-14, wherein the one or more genes that are upregulated in carcinoma-associated fibroblasts are TGFBR1 and/or STAT3.

16. The method of any one of claims 5-15, wherein the cancerous tumor is a colorectal cancer (CRC), a breast cancer, or a melanoma.

17. The method of any one of claims 5-16, wherein the subject is a human.

18. A method of inhibiting extracellular matrix (ECM) remodeling in the microenvironment of a cancerous tumor, which comprises administering to the microenvironment of a cancerous tumor a composition comprising one or more extracellular matrix crosslinking proteins and a pharmaceutically acceptable carrier, whereby ECM remodeling in the microenvironment of the cancerous tumor is inhibited.

19. The method of claim 18, wherein the one or more ECM crosslinking proteins are HAPLN1, genipin, and/or tissue transglutaminase (TG2).

20. The method of claim 18 or claim 19, wherein the cancerous tumor is a colorectal cancer (CRC), a breast cancer, or a melanoma.

21. The method of any one of claims 18-20, wherein the method is performed in vivo.

22. The method of claim 21, wherein the method is performed in a human.

23. The method of any one of claims 18-20, wherein the method is performed ex vivo.
